SEQUENCE LISTING (10 sequences)

SEQ ID NO: 1 - DNA, 1443 bp, Bacteroides fragilis
  source 1..1443: mol_type = other DNA
atggaagatttaaatttcagaaaaggtgatgctaaaacagaagcatttggttcaaacagaatgttacaaccctctccggtagagaaaatacctgatggtcctactactccggaaatcgcctatcagatggtgaaggacgaaacttttgctcaaactcagccccgtctgaatcttgctacattcgtgactacttatatggatgattatgcaacgaagctgatgaacgaagctatcaatatcaactacattgatgagacagaatatcctcgcattgctgtgatgaacggtaaatgtatcaatatcgttgctaatttgtggaactctccggaaaaagatacctggaaaaccggtgcattggctatcggttcttcagaagcttgtatgttgggtggtgtagctgcctggttgcgttggcgcaaaaaacgtcaggcccagggtaaaccatttgataaacctaactttgtcatttcaaccggtttccaggttgtttgggaaaaatttgctcagttgtggcagattgagatgcgtcaggtgcctttgacgctggacaagaccacacttgacccggaagaagccctgaagatgtgtgatgaaaatacaatctgtgtagttccaatccaaggggttacatggaccggtctgaacgatgatgttgaagcccttgacaaagccctcgatgcgtataacgctaaaaccggttatgatattcctattcacgtagacgctgccagcggtggtttcatcctgccgttcctgtatcctgataccaaatgggacttccgtctgaaatgggttctttccatcagtgtatccggccataagttcggtctcgtatatccgggtttgggttgggttgtctggaaaggcaaagaatatctgcccgaagaaatggctttcagcgtaaactacttaggagctaacattactcaggtaggtttgaacttctctcgtcctgcagctcagattttgggacaatattatcaatttattcgtttaggattccagggatacaaggaagtacaatacaactctttgcagattgccaaatacatccacagccagattgctaagatgactccgttcgtcaactactcggaagatgtagtaaacccgttgttcatttggtacatgaagccggaatatgcaaagaacgccaaatggactctttacgatttgcaggataagctggctcagcatggctggatggttccggcatatacattgcctgccaagctgcaagattatgtggttatgcgtgtcgttgtccgtcagggattcagtcgtgatatggccgacatgttgctgggcgacattaagaatgctattgccgaactggaaaaactggaatacccgacatccactcgtattgcccaggagaagaatctgccggtagaagccaaagtatttaaccataccggtaaaccacaagctgccaagaaataa

SEQ ID NO: 2 - protein, 480 aa, Bacteroides fragilis
MEDLNFRKGDAKTEAFGSNRMLQPSPVEKIPDGPTTPEIAYQMVKDETFAQTQPRLNLATFVTTYMDDYATKLMNEAININYIDETEYPRIAVMNGKCINIVANLWNSPEKDTWKTGALAIGSSEACMLGGVAAWLRWRKKRQAQGKPFDKPNFVISTGFQVVWEKFAQLWQIEMRQVPLTLDKTTLDPEEALKMCDENTICVVPIQGVTWTGLNDDVEALDKALDAYNAKTGYDIPIHVDAASGGFILPFLYPDTKWDFRLKWVLSISVSGHKFGLVYPGLGWVVWKGKEYLPEEMAFSVNYLGANITQVGLNFSRPAAQILGQYYQFIRLGFQGYKEVQYNSLQIAKYIHSQIAKMTPFVNYSEDVVNPLFIWYMKPEYAKNAKWTLYDLQDKLAQHGWMVPAYTLPAKLQDYVVMRVVVRQGFSRDMADMLLGDIKNAIAELEKLEYPTSTRIAQEKNLPVEAKVFNHTGKPQAAKK

SEQ ID NO: 3 - DNA, 1443 bp, synthetic construct
  source 1..1443: mol_type = other DNA
atggaagatttaaatttcagaaaaggtgatgctaaaacagaagcatttggttcaaacagaatgttacaaccctctccggtagagaaaatacctgatggtcctactactccggaaatcgcctatcagatggtgaaggacgaaacttttgctcaaactcagccccgtctgaatcttgctacattcgtgactacttatatggatgattatgcaacgaagctgatgaacgaagctatcaatatcaactacattaatgagacagaatatcctcgcattgctgtgatgaacggtaaatgtatcaatatcgttgctaatttgtggaactctccggaaaaagatacctggaaaaccggtgcattggctatcggttcttcagaagcttgtatgttgggtggtgtagctgcctggttgcgttggcgcaaaaaacgtcaggcccagggtaaaccatttgataaacctaactttgtcatttcaaccggtttccaggttgtttgggaaaaatttgctcagttgtggcagattgagatgcgtcaggtgcctttgacgctggacaagaccacacttgacccggaagaagccctgaagatgtgtgatgaaaatacaatctgtgtagttccaatccaaggggttacatggaccggtctgaacgatgatgttgaagcccttgacaaagccctcgatgcgtataacgctaaaaccggttatgatattcctattcacgtagacgctgccagcggtggtttcatcctgccgttcctgtatcctgataccaaatgggacttccgtctgaaatgggttctttccatcagtgtatccggccataagttcggtctcgtatatccgggtttgggttgggttgtctggaaaggcaaagaatatctgcccgaagaaatggctttcagcgtaaactacttaggagctaacattactcaggtaggtttgaacttctctcgtcctgcagctcagattttgggacaatattatcaatttattcgtttaggattccagggatacaaggaagtacaatacaactctttgcagattgccaaatacatccacagccagattgctaagatgactccgttcgtcaactactcggaagatgtagtaaacccgttgttcatttggtacatgaagccggaatatgcaaagaacgccaaatggactctttacgatttgcaggataagctggctcagcatggctggatggttccggcatatacattgcctgccaagctgcaagattatgtggttatgcgtgtcgttgtccgtcagggattcagtcgtgatatggccgacatgttgctgggcgacattaagaatgctattgccgaactggaaaaactggaatacccgacatccactcgtattgcccaggagaagaatctgccggtagaagccaaagtatttaaccataccggtaaaccacaagctgccaagaaataa

SEQ ID NO: 4 - protein, 480 aa, synthetic construct
MEDLNFRKGDAKTDVFGSNKMLQPSPVEKIPDGPTTPEIAYQMVKDETFAQTQPRLNLATFVTTYMDDYATKLMNEAININYINETEYPRIAVMNGKCINIVANLWNSPEKDTWKTGALAIGSSEACMLGGVAAWLRWRKKRQAQGKPFDKPNFVISTGFQVVWEKFAQLWQIEMRQVPLTLDKTTLDPEEALKMCDENTICVVPIQGVTWTGLNDDVEALDKALDAYNAKTGYDIPIHVDAASGGFILPFLYPETKWDFRLKWVLSISVSGHKFGLVYPGLGWVVWKGKEYLPEEMAFSVNYLGANITQVGLNFSRPAAQILGQYYQFIRLGFQGYKEVQYNSLQIAKYIHSQIAKMAPFVNYSEDVVNPLFIWYMKPEYAKNAKWTLYDLQDKLAQHGWMVPAYTLPSKLENYVVMRVVVRQGFSWDMADMLLGDIKNAITELEKLEYPTTTRIAQEKNLPVETKVFNHTGKPQVAKK

SEQ ID NO: 5 - DNA, 1443 bp, synthetic construct
  source 1..1443: mol_type = other DNA
atggaagatttaaatttcagaaaaggtgatgctaaaacagaagcatttggttcaaacagaatgttacaaccctctccggtagagaaaatacctgatggtcctactactccggaaatcgcctatcagatggtgaaggacgaaacttttgctcaaactcagccccgtctgaatcttgctacatgggtgactacttatatggatgattatgcaacgaagctgatgaacgaagctatcaatatcaactacattgatgagacagaatatcctcgcattgctgtgatgaacggtaaatgtatcaatatcgttgctaatttgtggaactctccggaaaaagatacctggaaaaccggtgcattggctatcggttcttcagaagcttgtatgttgggtggtgtagctgcctggttgcgttggcgcaaaaaacgtcaggcccagggtaaaccatttgataaacctaactttgtcatttcaaccggtttccaggttgtttgggaaaaatttgctcagttgtggcagattgagatgcgtcaggtgcctttgacgctggacaagaccacacttgacccggaagaagccctgaagatgtgtgatgaaaatacaatctgtgtagttccaatccaaggggttacatggaccggtctgaacgatgatgttgaagcccttgacaaagccctcgatgcgtataacgctaaaaccggttatgatattcctattcacgtagacgctgccagcggtggtttcatcctgccgttcctgtatcctgataccaaatgggacttccgtctgaaatgggttctttccatcagtgtatccggccataagttcggtctcgtatatccgggtttgggttgggttgtctggaaaggcaaagaatatctgcccgaagaaatggctttcagcgtaaactacttaggagctaacattactcaggtaggtttgaacttctctcgtcctgcagctcagattttgggacaatattatcaatttattcgtttaggattccagggatacaaggaagtacaatacaactctttgcagattgccaaatacatccacagccagattgctaagatgactccgttcgtcaactactcggaagatgtagtaaacccgttgttcatttggtacatgaagccggaatatgcaaagaacgccaaatggactctttacgatttgcaggataagctggctcagcatggctggatggttccggcatatacattgcctgccaagctgcaagattatgtggttatgcgtgtcgttgtccgtcagggattcagtcgtgatatggccgacatgttgctgggcgacattaagaatgctattgccgaactggaaaaactggaatacccgacatccactcgtattgcccaggagaagaatctgccggtagaagccaaagtatttaaccataccggtaaaccacaagctgccaagaaataa

SEQ ID NO: 6 - protein, 480 aa, synthetic construct
MEDLNFRKGDAKTDVFGSNKMLQPSPVEKIPDGPTTPEIAYQMVKDETFAQTQPRLNLATWVTTYMDDYATKLMNEAININYIDETEYPRIAVMNGKCINIVANLWNSPEKDTWKTGALAIGSSEACMLGGVAAWLRWRKKRQAQGKPFDKPNFVISTGFQVVWEKFAQLWQIEMRQVPLTLDKTTLDPEEALKMCDENTICVVPIQGVTWTGLNDDVEALDKALDAYNAKTGYDIPIHVDAASGGFILPFLYPETKWDFRLKWVLSISVSGHKFGLVYPGLGWVVWKGKEYLPEEMAFSVNYLGANITQVGLNFSRPAAQILGQYYQFIRLGFQGYKEVQYNSLQIAKYIHSQIAKMAPFVNYSEDVVNPLFIWYMKPEYAKNAKWTLYDLQDKLAQHGWMVPAYTLPSKLENYVVMRVVVRQGFSWDMADMLLGDIKNAITELEKLEYPTTTRIAQEKNLPVETKVFNHTGKPQVAKK

SEQ ID NO: 7 - DNA, 41 bp, synthetic construct
  source 1..41: mol_type = other DNA
ccgcaatgcgcggatattcggtttcattaatatagttaatg

SEQ ID NO: 8 - DNA, 41 bp, synthetic construct
  source 1..41: mol_type = other DNA
cattaactatattaatgaaaccgaatatccgcgcattgcgg

SEQ ID NO: 9 - DNA, 42 bp, synthetic construct
  source 1..42: mol_type = other DNA
catccatataggtggtcacccaggtcgccaggttcaggcgcg

SEQ ID NO: 10 - DNA, 42 bp, synthetic construct
  source 1..42: mol_type = other DNA
cgcgcctgaacctggcgacctgggtgaccacctatatggatg

(19) United States
(12) Patent Application Publication
(10) Pub. No.: US 2026/0234597 A1
(43) Pub. Date: Aug. 13, 2026

(54) SYNTHESIS OF VERSATILE NEUROMODULATORY MOLECULES BY A GUT MICROBIAL GLUTAMATE DECARBOXYLASE

(71) Applicants: Dhara D. SHAH, Chandler, AZ (US); Pavani DADI, Tempe, AZ (US)

(72) Inventors: Dhara D. SHAH, Chandler, AZ (US); Pavani DADI, Tempe, AZ (US)

(21) Appl. No.: 19/066,581

(22) Filed: Feb. 28, 2025

Related U.S. Application Data
(60) Provisional application No. 63/559,331, filed on Feb. 29, 2024.

(51) Int. Cl. C12N 9/88; C12N 1/20; C12N 15/70; C12P 13/00; C12P 13/06

(52) U.S. Cl. CPC C12N 9/88; C12N 1/20; C12N 15/70; C12P 13/005; C12P 13/06; C12Y 401/01015

(57) ABSTRACT

Described herein are isolated polypeptides comprising a Bacteroides fragilis glutamate decarboxylase (BfGAD) mutant having one or more amino acid substitutions introduced into a wild-type BfGAD polypeptide sequence, where the BfGAD mutant provides an improvement in decarboxylase activity as compared to the wild-type BfGAD polypeptide. Also described herein are methods for synthesizing neuromodulatory molecules comprising contacting a BfGAD mutant with a substrate.

### CROSS-REFERENCE TO RELATED APPLICATIONS

This application claims priority to U.S. Provisional Patent Application No. 63/559,331, filed on Feb. 29, 2024, which is incorporated by reference herein in its entirety.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “208192-0015-US02_sequence_listing_xml_25 Feb. 2025.xml,” was created on Feb. 25, 2025, contains 10 sequences, has a file size of 16.0 kbytes (16,384 bytes), and is incorporated by reference in its entirety into the specification.

### BACKGROUND

Low levels of inhibitory neurotransmitter like gamma-aminobutyric acid (GABA) have been associated with many different neurological disorders such as epilepsy, schizophrenia, autism spectrum disorder (ASD), ADHD, panic disorder, PTSD, major depressive disorder, progressive multiple sclerosis, dementia, and Alzheimer's disease. Besides GABA, taurine is another amino acid that is found in abundance in the brain. Taurine is a GABAA receptor agonist and exerts downstream effects similar to GABA. Concentrations of taurine are altered in individuals and mouse models of Alzheimer's disease, where reduced levels of taurine are found in the brain, while higher urinary excretion of taurine is seen in elderly patients with dementia. In a separate study, gnotobiotic mouse transplanted with fecal microbiota from an Alzheimer's patient showed lower abundance of GABA and taurine in the feces. Based on these observations, supplementation with taurine and its analogs has been investigated in Alzheimer's disease models and in patients. In one such study, Homotaurine, a molecule similar to taurine, was shown to restore cognitive functions in patients with Alzheimer's disease. In addition, it was also observed that taurine reverses cognitive deficits in APP/PS1 mouse model and improves learning and memory in mice.

Studies that correlate gut microbial dysbiosis to two of the prominent neurodegenerative disorders-Dementia and Alzheimer's disease, often show modulations in the abundance of microbes of the genus Bacteroides. Bacteroides are prominent members of the human gut and have the ability to produce GABA. All Bacteroides encode the gene for the enzyme glutamate decarboxylase (GAD). Glutamate decarboxylases (GADs) are PLP (pyridoxal phosphate) dependent enzymes, which catalyze conversion of an excitatory neurotransmitter glutamate to an inhibitory neurotransmitter GABA. However, very little is known about the chemistry of the GADs encoded by Bacteroides sp.

Despite widespread prevalence of substrate promiscuity in GADs across different domains of life, substrate specificity and product formation landscape for most prokaryotic GADs, specifically GADs of Bacteroides sp., are still not clearly understood.

What is needed are GAD variants having improved decarboxylase activity either towards glutamate or with the ability to produce multiple neuromodulatory molecules.

### SUMMARY

One embodiment described herein isan isolated polypeptide comprising a Bacteroides fragilis glutamate decarboxylase (BfGAD) mutant having one or more amino acid substitutions introduced into a wild-type BfGAD polypeptide sequence (SEQ ID NO: 2), where the BfGAD mutant provides an improvement in decarboxylase activity as compared to the wild-type BfGAD polypeptide sequence (SEQ ID NO: 2). In one aspect, the BfGAD mutant comprises one or more of the following substitutions: D104N or F81W relative to the wild-type BfGAD polypeptide sequence (SEQ ID NO: 2). In another aspect, the BfGAD mutant comprises an amino acid sequence having at least 95% identity to SEQ ID NO: 4 or SEQ ID NO: 6. In another aspect, the BfGAD mutant comprises the amino acid sequence of SEQ ID NO: 4 or SEQ ID NO: 6.

Another embodiment described herein is an isolated nucleotide encoding a Bacteroides fragilis glutamate decarboxylase (BfGAD) mutant having one or more amino acid substitutions introduced into a wild-type BfGAD polypeptide sequence (SEQ ID NO: 2). In one aspect, the BfGAD mutant comprises one or more of the following substitutions: D104N or F81W relative to the wild-type BfGAD polypeptide sequence (SEQ ID NO: 2). In another aspect, the isolated nucleotide comprises a polynucleotide sequence having at least 95% identity to SEQ ID NO: 3 or SEQ ID NO: 5. In another aspect, the isolated nucleotide comprises the polynucleotide sequence of SEQ ID NO: 3 or SEQ ID NO: 5.

Another embodiment described herein is an isolated nucleotide vector comprising an isolated nucleotide described herein.

Another embodiment described herein is a cell comprising an isolated nucleotide vector of described herein.

Another embodiment described herein is a method for synthesizing neuromodulatory molecules, the method comprising: contacting a Bacteroides fragilis glutamate decarboxylase (BfGAD) mutant with a substrate, and obtaining one or more neuromodulatory molecules. In one aspect, the BfGAD mutant comprises one or more of the following substitutions: D104N or F81W relative to a wild-type BfGAD polypeptide sequence (SEQ ID NO: 2). In another aspect, the BfGAD mutant comprises an amino acid sequence having at least 95% identity to SEQ ID NO: 4 or SEQ ID NO: 6. In another aspect, the BfGAD mutant comprises the amino acid sequence of SEQ ID NO: 4 or SEQ ID NO: 6. In another aspect, the neuromodulatory molecules comprise γ-aminobutyric acid (GABA), hypotaurine, taurine, homotaurine, or β-alanine. In another aspect, the substrate comprises L-glutamate, L-cysteate, L-homocysteate, or L-aspartate.

Another embodiment described herein is a neuromodulatory molecule comprising γ-aminobutyric acid (GABA), hypotaurine, taurine, homotaurine, or β-alanine synthesized by a method described herein.

Another embodiment described herein is a method for creating a Bacteroides fragilis strain with a deletion of the gene encoding glutamate decarboxylase (B. fragilis ΔGAD), the method comprising: (a) transforming a suicide vector encoding B. fragilis glutamate decarboxylase into competent E. coli cells and screening for transformants; (b) independently growing liquid cultures of transformed E. coli aerobically and growing liquid cultures of B. fragilis anaerobically; (c) independently isolating cells from the liquid cultures of the transformed E. coli and B. fragilis and resuspending the cells in a volume of liquid; (d) plating aliquots of each of the resuspended transformed E. coli and B. fragilis cells on a culture media plate and incubating aerobically at 37° C. overnight; (e) isolating B. fragilis cells from the culture media plate and streaking the cells on a second culture media plate comprising gentamicin and erythromycin and incubating the second culture media plate anaerobically at 37° C. for 2-3 days; (f) selecting individual B. fragilis colonies and analyzing the colonies for a deletion of the gene encoding glutamate decarboxylase; and (g) identifying a B. fragilis cell having a deletion of the gene encoding glutamate decarboxylase.

### DESCRIPTION OF THE DRAWINGS

FIG. 1A-D show characterization of BfGAD. FIG. 1A shows the possible role of gut microbes in producing neuromodulatory molecules. FIG. 1B shows spectra of PLP cofactor bound to the holoenzyme BfGAD_{WT} captured at various pH, depicting tautomers of internal aldimine at 335 nm and 420 nm. FIG. 1C shows a plot of initial velocity as a function of BfGAD_{WT} concentrations. FIG. 1D shows a plot of initial velocity vs L-glutamate concentrations. The solid line is a sigmoidal fit to the data.

FIG. 2A-F show catalytic and structural perturbations of evolved BfGADs. FIG. 2A shows the structure of E. coli GAD (1XEY) aligned with BfGAD showing point substitution D104N* (*residue from the neighboring monomer). FIG. 2B shows the structure of E. coli GAD (1XEY) aligned with BfGAD showing point substitution F81W. FIG. 2C shows the structure of BtGAD (7X51) aligned with BfGAD showing point substitution D104N* (*residue from the neighboring monomer). FIG. 2D shows the structure of BtGAD (7X51) aligned with BfGAD showing point substitution F81W. The dashed line indicates the hydrogen bond interactions of the residue with substrate analogue, glutarate (GUA). FIG. 2E shows the absorption spectra depicting 335 nm and 420 nm peaks of the bound PLP enzyme of BfGAD_{WT} and variants. FIG. 2F shows progress curves of BfGAD_{WT} and variants depicting the initial velocities (n=3).

FIG. 3A-F show headspace gas chromatography of wildtype and evolved BfGADs. FIG. 3A-F show chromatograms showing the CO₂ peaks obtained from the decarboxylation reaction of the substrates after 24 h incubation with BfGAD_{WT} (FIG. 3A), BfGAD_{D104N} (FIG. 3C), and BfGAD_{F81W} (FIG. 3E) using headspace GC. The CO₂ peak areas were obtained from the decarboxylation of the substrates after 24 h incubation with BfGAD_{WT} (FIG. 3B), BfGAD_{D104N} (FIG. 3D), and BfGAD_{F81W} (FIG. 3F).

FIG. 4A-D show thin layer chromatography with single substrates. FIG. 4A shows decarboxylation of L-CSA catalysed by BfGAD_{WT} and BfGAD_{D104N} in 24 h. FIG. 4B shows decarboxylation of L-CA catalysed by BfGAD_{WT} and BfGAD_{D104N} in 24 h. FIG. 4C shows decarboxylation of L-Aspartate (Asp) catalysed by BfGAD_{WT} and BfGAD_{D104N} in 24 h. FIG. 4D shows decarboxylation of L-HCA catalysed by BfGAD_{WT} and BfGAD_{D104N} in 24 h. The intensity plots from the TLC were analysed. The peaks are highlighted for the products (Hypotaurine, taurine, β-alanine, and Homotaurine) and for the substrates (L-CSA, L-CA, L-Asp, and L-HCA).

FIG. 5 shows thin layer chromatography of BfGAD_{WT} and BfGAD_{D104N} with mixed substrates. TLC analyses were performed at various timepoints of decarboxylation reactions of mixed substrate L-CSA (50 mM) with L-glutamate (5 mM), incubated with BfGAD_{WT} and BfGAD_{D104N}. Samples were collected at 3 h, 9 h, 24 h, 30 h, and 48 h. Standards of L-glutamate, L-CSA, GABA, and hypotaurine were run as references. Intensity plots obtained from the TLC plates were analysed. The highlighted peaks represent the product GABA, the product hypotaurine, the substrate L-CSA, and the substrate L-glutamate.

FIG. 6A shows thin layer chromatography of BfGAD_{WT} and BfGAD_{D104N} with mixed substrates. TLC analyses were performed at various timepoints of decarboxylation reactions of mixed substrate L-CA (50 mM) with L-glutamate (5 mM), incubated with BfGAD_{WT} and BfGAD_{D104N}. Samples were collected at 3 h, 9 h, 24 h, 30 h, and 48 h. Standards of L-glutamate, L-CA, GABA, and taurine were run as references. Intensity plots obtained from the TLC plates were analysed. The highlighted peaks represent the production of GABA, the production of taurine, the remaining level of L-CA, and the remaining level of L-glutamate over the time course.

FIG. 6B shows thin layer chromatography of BfGAD_{WT} and BfGAD_{D104N} with mixed substrates. TLC analysis was performed at various timepoints of decarboxylation reactions of mixed substrate L-Asp (50 mM) with L-glutamate (5 mM), incubated with BfGAD_{WT} and BfGAD_{D104N}. Samples were collected at 3 h, 9 h, 24 h, 30 h, and 48 h. Standards of L-glutamate, L-Asp, GABA, and β-alanine were run as references. Intensity plots obtained from the TLC plates were analysed. The highlighted peaks represent the production of GABA, the production of β-alanine, the remaining level of L-Asp, and the remaining level of L-glutamate over the time course.

FIG. 7A-B show hypotaurine (HT) and taurine detection via LC-MS/MS from BfGAD_{WT} and BfGAD_{D104N} catalyzed mixed substrate reactions. FIG. 7A shows ion chromatograms of product hypotaurine and the remaining L-CSA substrate in the mixed substrate reactions with a 1:10 ratio of L-glutamate:L-CSA catalyzed by BfGAD_{WT}. FIG. 7B shows ion chromatograms of taurine and remaining L-CA in the mixed substrate reactions with a 1:10 ratio of L-Glutamate:L-CA catalyzed by BfGAD_{D104N}.

FIG. 8 shows phylogenetic analysis of glutamate decarboxylases (GADs) from various species of Bacteroides.

FIG. 9 shows multiple sequence alignment of different GADs of human gut microbes, highlighting the conserved residues that are predicted to be important in substrate binding and cofactor (PLP) binding. Here, BfGAD residue numbers are shown without an N-terminal His tag.

FIG. 10 shows the phylogenetic analysis of GADs from various gut microbes, (BfGAD, highlighted). Top numbers of each branch represent branch lengths and bottom numbers of each branch represent bootstrap values.

FIG. 11A-B show purification of BfGAD_{WT}. FIG. 11A shows SDS-PAGE analysis of BfGAD throughout purification. M, molecular weight marker; CL, cell lysate; SP, supernatant of the sonicated cells; FT, flow-through from Ni-NTA loading; W, wash collected from the Ni-NTA column; and P, purified BfGAD. FIG. 11B shows native PAGE analysis of purified BfGAD. M, molecular weight marker; and P, purified BfGAD.

FIG. 12A-D show gel filtration of BfGAD_{WT}. FIG. 12A shows gel filtration chromatography of BfGAD at pH 7.2 and pH 4.7. Gel filtration analysis was performed in 50 mM Hepes buffer, pH 7.2 with 0.15 mM NaCl, and 50 mM sodium acetate buffer, pH 4.7 with 0.15 mM NaCl, at a flow rate of 0.5 mL/min, at 4° C. FIG. 12B shows a calibration curve of the protein standards from gel filtration chromatography (SEC). FIG. 12C shows a table representing the MW of the standards and the predicated MWs of BfGAD in buffers at pH 4.7 and at pH 7.2. FIG. 12D shows gel filtration chromatography of BfGAD in pH 4.7, with and without addition of external PLP.

FIG. 13A-B show the pH influence on activity. FIG. 13A shows the absorbance at 420 nm of BfGAD at different pH, fit to a simulated curve. FIG. 13B shows activity of BfGAD at different pH.

FIG. 14A-B show structures of GADA. FIG. 14A shows structures of E. coli GADA (1XEY) superimposed with E. coli GADB (1PMM). FIG. 14B shows the zoomed in active sites of EcGADs with superimposed structures of ligand bound E. coli GADA and E. coli GADB.

FIG. 15A-D show activity of wildtype and engineered BfGADs with L- and D-glutamate.

FIG. 15A shows the activity of BfGAD_{WT} with L- and D-glutamate. FIG. 15B shows the activity of BfGAD_{D104N} with L- and D-glutamate. FIG. 15C shows the activity of BfGAD_{F81W} with L- and D-glutamate. FIG. 15D shows thin layer chromatography (TLC) analysis of the reactions catalyzed by BfGAD_{WT} and engineered BfGADs incubated with D-glutamate.

FIG. 16A-F show peak areas of CO₂. The CO₂ peak areas after subtracting the peak areas from control reactions were obtained from the decarboxylation of the substrates after 24 h and 48 h incubation with BfGAD_{WT} (FIG. 16A), BfGAD_{D104N} (FIG. 16B), and BfGAD_{F81W} (FIG. 16C). The CO₂ peak areas of the same reactions along with no enzyme controls for BfGAD_{WT} (FIG. 16D), BfGAD_{D104N} (FIG. 16E), and BfGAD_{F81W} (FIG. 16F).

FIG. 17A-E show thin layer chromatography of BfGAD_{WT} and BfGAD_{D104N} with single substrates. FIG. 17A shows original TLC plates shown in FIG. 4A with analyzed decarboxylation reactions of L-CSA catalysed by BfGAD_{WT} and BfGAD_{D104N}. FIG. 17B shows original TLC plates shown in FIG. 4B with analyzed decarboxylation reactions of L-CA catalysed by BfGAD_{WT} and BfGAD_{D104N}. FIG. 17C shows original TLC plates shown in FIG. 4C with analyzed decarboxylation reactions of L-aspartate catalysed by BfGAD_{WT} and BfGAD_{D104N}. FIG. 17D shows original TLC plates shown in FIG. 4D with analyzed decarboxylation reactions of L-HCA catalysed by BfGAD_{WT} and BfGAD_{D104N}. FIG. 17E shows original single substrate reactions of L-CSA and L-CA with BfGAD_{WT} and BfGAD_{D104N} in 24 and 48 hours. Standards of L-CSA, L-CA, L-Asp, hypotaurine, taurine, and β-alanine were run as references.

FIG. 18A-B show thin layer chromatography of BfGAD_{F81W} with single substrates. FIG. 18A shows analysis of decarboxylation reactions of L-CSA and L-CA catalyzed by BfGAD_{F81W} via TLC. FIG. 18B shows analysis of decarboxylation reactions of L-Asp and L-HCA catalyzed by BfGAD_{F81W} via TLC. Standards of L-CSA, L-CA, L-Asp, hypotaurine, taurine, and β-alanine were run as references.

FIG. 19A-E show thin layer chromatography of BfGAD_{WT} and BfGAD_{D104N} with mixed substrates. Timepoint reactions of mixed substrates incubated with BfGAD_{WT} and BfGAD_{D104N}. FIG. 19A shows L-CSA (50 mM) with L-glutamate (5 mM) (1:10 L-glutamate: L-CSA ratio). FIG. 19B shows L-CA (50 mM) with L-glutamate (5 mM) (1:10 L-glutamate: L-CA ratio). FIG. 19C shows L-Asp (50 mM) with L-glutamate (5 mM) (1:10 L-glutamate: L-Asp ratio). FIG. 19D shows a 1:5 L-glutamate: L-Asp ratio. FIG. 19E shows a 1:5 L-glutamate: L-CSA ratio. Standards of L-glutamate, L-CSA, L-CA, L-Asp, GABA, hypotaurine, taurine, and β-alanine were run as references.

FIG. 20A-D show LC-MS/MS chromatograms of standards: L-CA (FIG. 20A), Taurine (FIG. 20B), L-CSA (FIG. 20C), and Hypotaurine (FIG. 20D).

FIG. 21 shows de novo biosynthesis of taurine with a central role of the enzyme cysteinesulfinic acid decarboxylase (CSAD).

FIG. 22A-B show CO₂ Standards. FIG. 22A shows chromatograms obtained from the headspace GC analysis of CO₂ standard with different volumes. FIG. 22B shows peak areas of injected CO₂ standards.

FIG. 23 shows thin layer chromatography (TLC) results for WT and BfΔGAD.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended”term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjuctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points, or as described above in the definition of “about.”

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

Dysbiosis of the microbiome correlates with many neurological disorders, yet very little is known about the chemistry that controls the production of neuromodulatory molecules by gut microbes. An enzyme glutamate decarboxylase of a gut microbe Bacteroides fragilis (BfGAD) produces multiple neuromodulatory molecules such as γ-aminobutyric acid (GABA), hypotaurine, taurine, homotaurine, and β-alanine. As described herein, the BfGAD has been evolved, resulting in two-fold increase in taurine productivity in one variant and enhanced specificity towards the substrate L-glutamate in another. A chemical strategy is described herein where BfGAD activity can be fine-tuned. This strategy can be used to modulate the production of neuromodulatory molecules by gut microbes.

The role of annotated GAD from Bacteroides fragilis was explored in the production of neuromodulatory molecules like GABA, taurine and its analogs, and β-alanine. The biochemical characterization of BfGAD showed kinetic and functional divergence of this enzyme from other studied prokaryotic GADs. The studies described herein show that BfGAD is not only specific to L-glutamate, but it can decarboxylate other substrates to produce multiple neuromodulatory molecules. Even within the mixture of substrates that includes L-glutamate, products beyond GABA were identified. This points towards the ability of this enzyme to function in a complex system with multiple substrates which can be a beneficial trait for microbes present in the human gut. Through rational protein engineering, a BfGAD variant capable of producing two-fold more taurine compared to the native BfGAD was created. Additionally, BfGAD was evolved to be more specific towards L-glutamate.

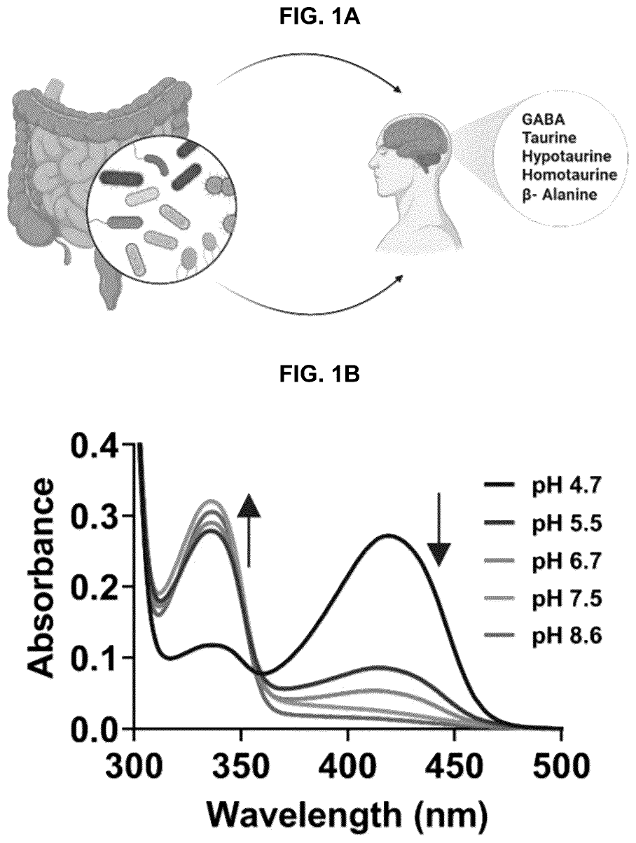

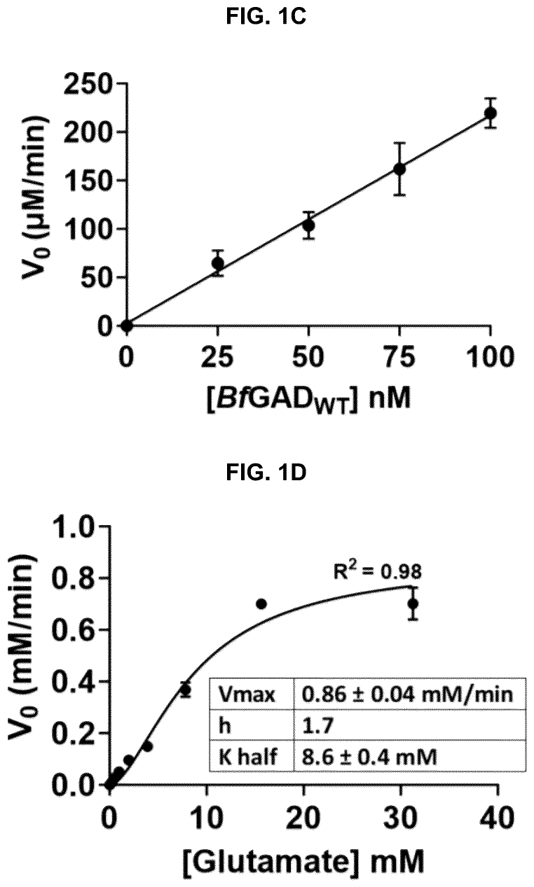

Based on the initial engineering results disclosed herein with BfGAD, the enzyme seems resilient in nature and is able to tolerate changes to the active site very well. It is envisioned that BfGAD can be evolved with rational designing and engineering, either to produce multiple neuromodulatory molecules in various proportions or to generate only a specific neuromodulatory molecule (FIG. 1A). This approach enables the generation of variants of BfGAD that can provide a road to therapeutic interventions for multiple neurodegenerative disorders.

Described herein are isolated polynucleotides and polypeptides, recombinant methods for producing the polynucleotides and polypeptides, codon-optimized polynucleotides for expressing the polypeptides, vectors containing the polynucleotides, expression systems for producing the polypeptides, and cultured host cells comprising such expression systems.

The polypeptides described herein can be provided in an isolated form. The term “polypeptide” encompasses “isolated polypeptide.” The phrase “isolated polypeptide” implies that a polypeptide is removed from its native environment. Thus, a polypeptide produced and/or contained within a recombinant cultured host cell is considered isolated for purposes described herein. Further, “isolated polypeptides” are polypeptides that have been purified, partially or substantially, from a recombinant cultured host. “Isolated polypeptides” also encompasses synthetically made polypeptides either by chemical synthesis or by recombinant methods.

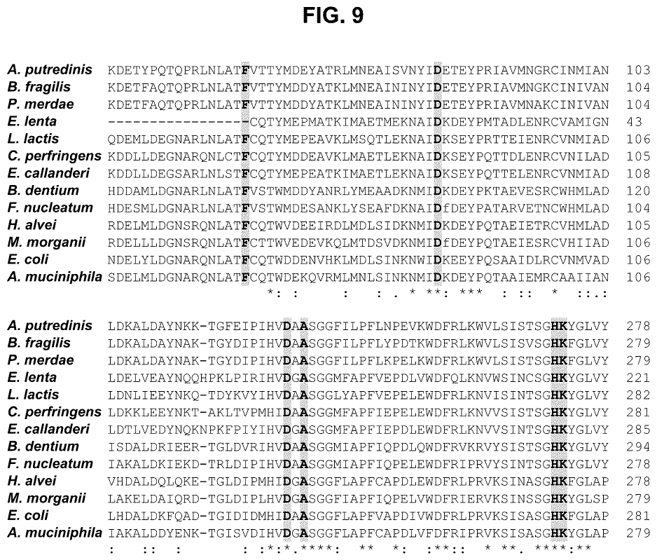

Sequence alignments such as those shown in FIG. 9, can be used to determine where substitutions or mutations can be made in the active site of the enzymes described herein to alter enzymatic activity or generate neomorphic activity. Further, substitutions or mutations can be made in order to modify the primary, secondary, or tertiary structure based on evolutionarily conserved residues as described herein. Such modifications can reduce or enhance enzymatic activity, alter the substrate or co-factor preference, or generate neomorphic enzymatic activity.

One embodiment described herein are polynucleotides encoding the polypeptides disclosed herein or a polypeptide having conservative amino acid substitutions thereof. Guidance regarding selection of “conservative” amino acid substitutions is provided in more detail below. In one embodiment, the polynucleotide is DNA.

Another aspect described herein is codon-optimized polynucleotides encoding the polypeptides disclosed herein. A codon optimized polynucleotide encodes a polypeptide, but the native codons are optimized for enhanced expression in the cells of an organism by replacing one or more, or a significant number, of codons of the native sequence with codons that are more frequently or most frequently used in the genes of that particular organism. Various species exhibit biases for particular codons of an amino acid. Species-specific codon tables and programs for codon optimization are available for creating codon optimized coding sequences of the polynucleotides described herein.

Another aspect described herein is isolated polynucleotides encoding the polypeptides disclosed herein. The phrase “isolated polynucleotide” implies that a polynucleotide is removed from its native environment. Thus, a polynucleotide produced and/or contained within a recombinant cultured host cell is considered isolated for purposes described herein. Further, “isolated polynucleotides” are polynucleotides that have been purified, partially or substantially, from a recombinant cultured host. In addition, isolated polynucleotides comprise polynucleotides that are produced by recombinant means, by methods such as PCR, synthetic meas, such as solid-phase synthesis, and any other means know in the art for isolating polynucleotides from their native environment.

Another aspect described herein is a method of making a vector comprising inserting the polynucleotides described herein into a vector. In another aspect, a vector produced by the method is described.

In another aspect, a method of making a cultured host cell comprising introducing the vector into a cultured host cell is described. In another aspect, a cultured host cell is produced by the methods described herein.

In another aspect, isolated polypeptides, produced by a method comprising: (a) introducing a vector comprising a polynucleotide encoding the polypeptide into a cultured host cell; (b) culturing the host cell; (c) expressing the polypeptide; and (d) recovering the polypeptide are described. In another aspect, a method for producing a polypeptide comprising: (a) culturing the host cell described herein under conditions where the vector is expressed; and (b) recovering the polypeptide is described.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, encoding polypeptides having the amino acid sequences in SEQ ID NO: 3 or 5; (b) nucleotide sequences, or degenerate, homologous, or codon-optimized variants thereof, encoding polypeptides having the amino acid sequences in SEQ ID NO: 3 or 5; and (c) nucleotide sequences capable of hybridizing to the complement of any of the nucleotide sequences in (a) or (b) above and capable of expressing functional polypeptides of amino acid sequences in SEQ ID NO: 3 or 5.

By a polynucleotide having a nucleotide sequence at least, for example, 90-99% “identical” to a reference nucleotide sequence encoding a B. fragilis GAD or mutant B. fragilis GAD is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding the B. fragilis GAD.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:4 82-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in SEQ ID NO: 3 or 5, or degenerate, homologous, or codon-optimized variants thereof, will encode a functional B. fragilis GAD.

The polynucleotides described herein include those encoding mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

Thus, fragments, derivatives, or analogs of the polypeptides of SEQ ID NO: 4 or 6 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an lgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of SEQ ID NO: 4 or 6 can be substituted with one or more conserved or non-conserved amino acid residue (preferably a conserved amino acid residue). In some cases, these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, 99% or 100% identical to the polypeptide sequence shown in SEQ ID NO: 4 or 6 and will comprise functional proteins or enzymes. Similarly, additions or deletions to the polypeptides can be made either at the N- or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1. Another embodiment described herein is a polynucleotide vector comprising one or more nucleotide sequences described herein.

Another embodiment described herein is a cell comprising one or more nucleotide sequences described herein or a polynucleotide vector described herein.

Another embodiment is a polypeptide encoded by a nucleotide sequence described herein. In one aspect, the polypeptide has 85% to 99% identity to SEQ ID NO: 4 or 6, including all integers, fractions end points, and subranges within the specified range. In another aspect, the polypeptide is selected from SEQ ID NO: 4 or 6.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the method or the means described herein

Another embodiment described herein is the use of an effective amount of a polypeptide encoded by one or more of the nucleotide sequences described herein in SEQ ID NO: 4 or 6.

Another embodiment described herein is a research tool comprising a polypeptide encoded by a nucleotide sequence described herein.

Another embodiment described herein is a reagent comprising a polypeptide encoded by a nucleotide sequence described herein.

One embodiment described herein isan isolated polypeptide comprising a Bacteroides fragilis glutamate decarboxylase (BfGAD) mutant having one or more amino acid substitutions introduced into a wild-type BfGAD polypeptide sequence (SEQ ID NO: 2), where the BfGAD mutant provides an improvement in decarboxylase activity as compared to the wild-type BfGAD polypeptide sequence (SEQ ID NO: 2). In one aspect, the BfGAD mutant comprises one or more of the following substitutions: D104N or F81W relative to the wild-type BfGAD polypeptide sequence (SEQ ID NO: 2). In another aspect, the BfGAD mutant comprises an amino acid sequence having at least 95% identity to SEQ ID NO: 4 or SEQ ID NO: 6. In another aspect, the BfGAD mutant comprises the amino acid sequence of SEQ ID NO: 4 or SEQ ID NO: 6.

Another embodiment described herein is an isolated nucleotide encoding a Bacteroides fragilis glutamate decarboxylase (BfGAD) mutant having one or more amino acid substitutions introduced into a wild-type BfGAD polypeptide sequence (SEQ ID NO: 2). In one aspect, the BfGAD mutant comprises one or more of the following substitutions: D104N or F81W relative to the wild-type BfGAD polypeptide sequence (SEQ ID NO: 2). In another aspect, the isolated nucleotide comprises a polynucleotide sequence having at least 95% identity to SEQ ID NO: 3 or SEQ ID NO: 5. In another aspect, the isolated nucleotide comprises the polynucleotide sequence of SEQ ID NO: 3 or SEQ ID NO: 5.

Another embodiment described herein is an isolated nucleotide vector comprising an isolated nucleotide described herein.

Another embodiment described herein is a cell comprising an isolated nucleotide vector of described herein.

Another embodiment described herein is a method for synthesizing neuromodulatory molecules, the method comprising: contacting a Bacteroides fragilis glutamate decarboxylase (BfGAD) mutant with a substrate, and obtaining one or more neuromodulatory molecules. In one aspect, the BfGAD mutant comprises one or more of the following substitutions: D104N or F81W relative to a wild-type BfGAD polypeptide sequence (SEQ ID NO: 2). In another aspect, the BfGAD mutant comprises an amino acid sequence having at least 95% identity to SEQ ID NO: 4 or SEQ ID NO: 6. In another aspect, the BfGAD mutant comprises the amino acid sequence of SEQ ID NO: 4 or SEQ ID NO: 6. In another aspect, the neuromodulatory molecules comprise γ-aminobutyric acid (GABA), hypotaurine, taurine, homotaurine, or β-alanine. In another aspect, the substrate comprises L-glutamate, L-cysteine sulfinate, L-cysteate, L-homocysteate, or L-aspartate.

Another embodiment described herein is a neuromodulatory molecule comprising γ-aminobutyric acid (GABA), hypotaurine, taurine, homotaurine, or β-alanine synthesized by a method described herein.

Another embodiment described herein is a method for creating a Bacteroides fragilis strain with a deletion of the gene encoding glutamate decarboxylase (B. fragilis ΔGAD), the method comprising: (a) transforming a suicide vector encoding B. fragilis glutamate decarboxylase into competent E. coli cells and screening for transformants; (b) independently growing liquid cultures of transformed E. coli aerobically and growing liquid cultures of B. fragilis anaerobically; (c) independently isolating cells from the liquid cultures of the transformed E. coli and B. fragilis and resuspending the cells in a volume of liquid; (d) plating aliquots of each of the resuspended transformed E. coli and B. fragilis cells on a culture media plate and incubating aerobically at 37° C. overnight; (e) isolating B. fragilis cells from the culture media plate and streaking the cells on a second culture media plate comprising gentamicin and erythromycin and incubating the second culture media plate anaerobically at 37° C. for 2-3 days; (f) selecting individual B. fragilis colonies and analyzing the colonies for a deletion of the gene encoding glutamate decarboxylase; and (g) identifying a B. fragilis cell having a deletion of the gene encoding glutamate decarboxylase.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. An isolated polypeptide comprising a Bacteroides fragilis glutamate decarboxylase (BfGAD) mutant having one or more amino acid substitutions introduced into a wild-type BfGAD polypeptide sequence (SEQ ID NO: 2), where the BfGAD mutant provides an improvement in decarboxylase activity as compared to the wild-type BfGAD polypeptide sequence (SEQ ID NO: 2).
Clause 2. The isolated polypeptide of clause 1, wherein the BfGAD mutant comprises one or more of the following substitutions: D104N or F81W relative to the wild-type BfGAD polypeptide sequence (SEQ ID NO: 2).
Clause 3. The isolated polypeptide of clause 1 or 2, wherein the BfGAD mutant comprises an amino acid sequence having at least 95% identity to SEQ ID NO: 4 or SEQ ID NO: 6.
Clause 4. The isolated polypeptide of clause 1, wherein the BfGAD mutant comprises the amino acid sequence of SEQ ID NO: 4 or SEQ ID NO: 6.
Clause 5. An isolated nucleotide encoding a Bacteroides fragilis glutamate decarboxylase (BfGAD) mutant having one or more amino acid substitutions introduced into a wild-type BfGAD polypeptide sequence (SEQ ID NO: 2).
Clause 6. The isolated nucleotide of clause 5, wherein the BfGAD mutant comprises one or more of the following substitutions: D104N or F81W relative to the wild-type BfGAD polypeptide sequence (SEQ ID NO: 2).
Clause 7. The isolated nucleotide of clause 5 or 6, wherein the isolated nucleotide comprises a polynucleotide sequence having at least 95% identity to SEQ ID NO: 3 or SEQ ID NO: 5.
Clause 8. The isolated nucleotide of any one of clauses 5-7, wherein the isolated nucleotide comprises the polynucleotide sequence of SEQ ID NO: 3 or SEQ ID NO: 5.
Clause 9. An isolated nucleotide vector comprising the isolated nucleotide of any one of clauses 5-8.
Clause 10. A cell comprising the isolated nucleotide vector of clause 9.
Clause 11. A method for synthesizing neuromodulatory molecules, the method comprising:
   contacting a Bacteroides fragilis glutamate decarboxylase (BfGAD) mutant with a substrate, and
   obtaining one or more neuromodulatory molecules.
Clause 12. The method of clause 11, wherein the BfGAD mutant comprises one or more of the following substitutions: D104N or F81W relative to a wild-type BfGAD polypeptide sequence (SEQ ID NO: 2).
Clause 13. The method of clause 11 or 12, wherein the BfGAD mutant comprises an amino acid sequence having at least 95% identity to SEQ ID NO: 4 or SEQ ID NO: 6.
Clause 14. The method of any one of clauses 11-13, wherein the BfGAD mutant comprises the amino acid sequence of SEQ ID NO: 4 or SEQ ID NO: 6.
Clause 15. The method of any one of clauses 11-14, wherein the neuromodulatory molecules comprise γ-aminobutyric acid (GABA), hypotaurine, taurine, homotaurine, or β-alanine.
Clause 16. The method of any one of clauses 11-15, wherein the substrate comprises L-glutamate, L-cysteine sulfinate, L-cysteate, L-homocysteate, or L-aspartate.
Clause 17. A neuromodulatory molecule comprising γ-aminobutyric acid (GABA), hypotaurine, taurine, homotaurine, or β-alanine synthesized by the method of any one of clauses 11-16.
Clause 18. A method for creating a Bacteroides fragilis strain with a deletion of the gene encoding glutamate decarboxylase (B. fragilis ΔGAD), the method comprising:
   (a) transforming a suicide vector encoding B. fragilis glutamate decarboxylase into competent E. coli cells and screening for transformants;
   (b) independently growing liquid cultures of transformed E. coli aerobically and growing liquid cultures of B. fragilis anaerobically;
   (c) independently isolating cells from the liquid cultures of the transformed E. coli and B. fragilis and resuspending the cells in a volume of liquid;
   (d) plating aliquots of each of the resuspended transformed E. coli and B. fragilis cells on a culture media plate and incubating aerobically at 37° C. overnight;
   (e) isolating B. fragilis cells from the culture media plate and streaking the cells on a second culture media plate comprising gentamicin and erythromycin and incubating the second culture media plate anaerobically at 37° C. for 2-3 days;
   (f) selecting individual B. fragilis colonies and analyzing the colonies for a deletion of the gene encoding glutamate decarboxylase; and
   (g) identifying a B. fragilis cell having a deletion of the gene encoding glutamate decarboxylase.

### EXAMPLES

### Example 1

### Materials

Kanamycin, IPTG, GABase from Pseudomonas fluorescens, β-ME (beta-mercaptoethanol), α-ketoglutarate, D-glutamic acid, L-cysteine sulfinic acid monohydrate, L-cysteic acid monohydrate, GABA (γ-aminobutyric acid), hypotaurine, taurine, L-aspartic acid sodium salt, β-alanine, HEPES, Imidazole, pyridoxal 5′-phosphate monohydrate, and TLC Silica gel 60 F254 (20 cm×20 cm) were purchased from Sigma-Aldrich. Sodium L-glutamate monohydrate, Luria-Bertani Broth (LB), buffer components, Sodium chloride, Sodium acetate, ninhydrin, and NADP⁺ disodium salt were purchased from Fisher Scientific. All restriction enzymes and competent cells of E. coli BL21 (DE3) and E. coli NEB5a were purchased from New England Biolabs (NEB). Headspace vials were purchased from Chemglass Inc.

### Experimental Procedures

The wild type B. fragilis glutamate decarboxylase nucleotide and polypeptide sequences are shown in Table 1.

TABLE 1
Wild type B. fragilis Glutamate Decarboxylase Sequences

B. fragilis Glutamate Decarboxylase Nucleotide Sequence (SEQ ID NO: 1)
ATGGAAGATTTAAATTTCAGAAAAGGTGATGCTAAAACAGAAGCATTTGGTTCAAACAGAATGTTACAACCCTCTC
CGGTAGAGAAAATACCTGATGGTCCTACTACTCCGGAAATCGCCTATCAGATGGTGAAGGACGAAACTTTTGCTCA
AACTCAGCCCCGTCTGAATCTTGCTACATTCGTGACTACTTATATGGATGATTATGCAACGAAGCTGATGAACGAA
GCTATCAATATCAACTACATTGATGAGACAGAATATCCTCGCATTGCTGTGATGAACGGTAAATGTATCAATATCG
TTGCTAATTTGTGGAACTCTCCGGAAAAAGATACCTGGAAAACCGGTGCATTGGCTATCGGTTCTTCAGAAGCTTG
TATGTTGGGTGGTGTAGCTGCCTGGTTGCGTTGGCGCAAAAAACGTCAGGCCCAGGGTAAACCATTTGATAAACCT
AACTTTGTCATTTCAACCGGTTTCCAGGTTGTTTGGGAAAAATTTGCTCAGTTGTGGCAGATTGAGATGCGTCAGG
TGCCTTTGACGCTGGACAAGACCACACTTGACCCGGAAGAAGCCCTGAAGATGTGTGATGAAAATACAATCTGTGT
AGTTCCAATCCAAGGGGTTACATGGACCGGTCTGAACGATGATGTTGAAGCCCTTGACAAAGCCCTCGATGCGTAT
AACGCTAAAACCGGTTATGATATTCCTATTCACGTAGACGCTGCCAGCGGTGGTTTCATCCTGCCGTTCCTGTATC
CTGATACCAAATGGGACTTCCGTCTGAAATGGGTTCTTTCCATCAGTGTATCCGGCCATAAGTTCGGTCTCGTATA
TCCGGGTTTGGGTTGGGTTGTCTGGAAAGGCAAAGAATATCTGCCCGAAGAAATGGCTTTCAGCGTAAACTACTTA
GGAGCTAACATTACTCAGGTAGGTTTGAACTTCTCTCGTCCTGCAGCTCAGATTTTGGGACAATATTATCAATTTA
TTCGTTTAGGATTCCAGGGATACAAGGAAGTACAATACAACTCTTTGCAGATTGCCAAATACATCCACAGCCAGAT
TGCTAAGATGACTCCGTTCGTCAACTACTCGGAAGATGTAGTAAACCCGTTGTTCATTTGGTACATGAAGCCGGAA
TATGCAAAGAACGCCAAATGGACTCTTTACGATTTGCAGGATAAGCTGGCTCAGCATGGCTGGATGGTTCCGGCAT
ATACATTGCCTGCCAAGCTGCAAGATTATGTGGTTATGCGTGTCGTTGTCCGTCAGGGATTCAGTCGTGATATGGC
CGACATGTTGCTGGGCGACATTAAGAATGCTATTGCCGAACTGGAAAAACTGGAATACCCGACATCCACTCGTATT
GCCCAGGAGAAGAATCTGCCGGTAGAAGCCAAAGTATTTAACCATACCGGTAAACCACAAGCTGCCAAGAAATAA
B. fragilis Glutamate Decarboxylase Polypeptide Sequence (SEQ ID NO: 2)
MEDLNFRKGDAKTEAFGSNRMLQPSPVEKIPDGPTTPEIAYQMVKDETFAQTQPRLNLATFVTTYMDDYATKLMNE
AININYIDETEYPRIAVMNGKCINIVANLWNSPEKDTWKTGALAIGSSEACMLGGVAAWLRWRKKRQAQGKPFDKP
NFVISTGFQVVWEKFAQLWQIEMRQVPLTLDKTTLDPEEALKMCDENTICVVPIQGVTWTGLNDDVEALDKALDAY
NAKTGYDIPIHVDAASGGFILPFLYPDTKWDFRLKWVLSISVSGHKFGLVYPGLGWVVWKGKEYLPEEMAFSVNYL
GANITQVGLNFSRPAAQILGQYYQFIRLGFQGYKEVQYNSLQIAKYIHSQIAKMTPFVNYSEDVVNPLFIWYMKPE
YAKNAKWTLYDLQDKLAQHGWMVPAYTLPAKLQDYVVMRVVVRQGFSRDMADMLLGDIKNAIAELEKLEYPTSTRI
AQEKNLPVEAKVFNHTGKPQAAKK

### Gene Cloning

A synthesized gene for B. fragilis glutamate decarboxylase (BfGAD) was created with the help of Genewiz from Azenta life sciences. The gene was codon optimized for expression in E. coli cells, which was then subcloned into a pET28a expression vector between the Ndel and HindIII restriction sites to incorporate an N-terminal hexahistidine (His6) Tag. The pET28a-BfGAD construct was confirmed by agarose gel electrophoresis, restriction digestion, sanger sequencing (Genewiz), and full plasmid sequencing (Plasmidsaurus), and then transformed into E. coli NEB5a and E. coli BL21 (DE3) competent cells by heat shock. Glycerol stocks of the cells harboring the construct pET28a-BfGAD were stored at −80° C.

### Expression and Purification of BfGAD_{WT} and Engineered BfGADs

E. coli BL21 (DE3) cells containing the pET28a-BfGADs were cultivated in LB medium containing kanamycin (50 μg/mL) with shaking (200 rpm) at 37° C. until OD600 reached 0.7. At this point, the BfGAD expression was induced with isopropyl β-D-1-thiogalactopyranoside (IPTG) to a final concentration of 1 mM and growth was continued at 37° C. for 3 hours to allow for protein expression with continuous shaking at 200 rpm. Cells were harvested via centrifugation at 4000×g for 30 min at 4° C. and cell pellets were stored at −80° C. The frozen cell pellets were thawed on ice and resuspended in buffer A containing 20 mM Tris-HCl, 500 mM NaCl, 40 mM imidazole, 0.1 mM PLP, pH 7.8. All subsequent purification steps were carried out at 4° C. The cells were disrupted by sonication (20000 Hz for 10 cycles of 30 seconds each altering 1 minute on ice) and centrifuged at 13500×g at 4° C. for 30 min to separate supernatant from cell debris. The supernatant was then filtered through 0.2 μm PES filter membrane and loaded onto a HisTrap HP column (5 mL, 1.6×2.5 cm, Ni Sepharose High-performance column, GE Healthcare, now Cytiva) pre-equilibrated with buffer A at 1 mL/min. The wash step was carried out by running 5-column volumes of lysis buffer A to elute contaminating proteins. BfGAD was eluted with a linear gradient from Buffer A (containing 40 mM imidazole) to an elution Buffer B containing 20 mM Tris-HCl buffer, 500 mM NaCl, 400 mM imidazole, 0.1 mM PLP, PH 7.8 at 2 mL/min. Eluted protein peaks were pooled, buffer exchanged and concentrated to a final buffer (50 mM HEPES, pH 7.2) using Vivaspin 50 kDa MWCO filters (Cytiva). Aliquots of purified proteins were stored at −80° C. SDS-PAGE analysis and activity assays were performed to confirm the purity and presence of functional BfGAD proteins.

Gel Filtration Chromatography with BfGAD_{WT}

To understand oligomeric state of BfGAD, gel filtration chromatography was performed at two different pH values, pH 4.7 (50 mM sodium acetate, 0.15 mM NaCl) and pH 7.2 (50 mM HEPES, 0.15 mM NaCl) using HiPrep™ 16/60 Sephacryl® S-200 HR column. Equilibration and elution steps were carried out at a flow rate of 0.5 mL/min. High molecular weight (HMW) and Low molecular weight (LMW) calibration kits (Cytiva) were used for column calibration, to create a standard curve, and for molecular weight determination.

### Activity Assays

BfGAD activity assays were performed by a coupled enzyme assay with a GABase system to measure GABA production spectrophotometrically. Briefly, BfGAD was incubated with 50 mM L-glutamate in 50 mM sodium acetate, pH 4.7 buffer or in buffers at other pH values for one hour at 25° C. Enzymatic reactions were stopped by boiling samples for 15 min. These samples were then centrifuged at 6000×g for 5 min and if necessary, dilutions were created in 50 mM Tris-HCl, pH 8.6. For GABA measurement, 75 μL of samples (either diluted or undiluted) were added to 25 μL of Gabase assay mix containing 10 mM BME, 2 mM α-ketoglutarate, 600 μM NADP+, and 30 μg (0.015 U/mL) of GABase in 50 mM Tris-HCl, pH 8.6. In the GABase assay, GABA is converted to succinic semialdehyde (SSA) and then to succinate with subsequent production of NADPH which was measured at 340 nm. Using an extinction coefficient of 6,220 M⁻¹ cm⁻¹ at 340 nm, NADPH concentrations were calculated which will provide GABA concentrations in measured samples ([NADPH]=[GABA]).

pH Variation Experiments with Absorbance Spectrophotometry and Activity Assays (GABA Production)

Spectra of BfGAD_{WT} were recorded from 200-800 nm in buffers at pH 4.7, 5.6, 6.7, 7.5, and 8.6 at 25° C. on Agilent Cary 3500 UV-Vis spectrophotometer. From these spectra, absorbance changes at 335 nm (enolimine) and 20 nm (ketoenmine) for enzyme bound PLP cofactor were collected. A plot of pH vs absorbance at 420 nm was generated and the curve fitting was carried out using Equation 1:

$$\frac{Abs HnE - Abs E}{Abs - Abs E} - 1 = \frac{[ {10}^{- npK} ]}{[ {10}^{- npH} ]} ,$$

where pK is the acid dissociation constant, n indicates the number of protons involved in the titration, and AbsHnE and AbsE are the absorbances at acidic and basic pH which produce protonated and deprotonated forms of the enzymes. The initial activity assays for the BfGAD_{WT} were performed in buffers at pH 4.7-8 with GABase assay system as mentioned in the above method. Based on the results of these activity assays, all subsequent enzymatic assays were carried out at pH 4.7.

### Steady-State Kinetics

Kinetic parameters of BfGAD_{WT} were determined by varying BfGAD concentrations (0.5 μM-2 μM) and by varying L-glutamate concentrations (0.125 mM-32 mM) in 50 mM sodium acetate, pH 4.7 at 25° C. For enzyme concentration variation experiments, reaction samples with each enzyme concentration were collected at 1 min time point after starting the reaction and then stopped by boiling for 15 min. Once cooled to room temperature, these samples were analyzed for GABA content with GABase assay as mentioned above. For the substrate concentration variation experiments, 100 μL aliquots of reactions with each substrate concentration were collected at different time intervals (0.2-30 min). The reactions were stopped by boiling the samples for 15 minutes. Once cooled to room temperature, these samples were analyzed for GABA content with GABase assay as mentioned above. The initial velocities of various reactions were calculated by fitting data of early timepoints to a linear regression. These initial velocities were then plotted against substrate concentrations and curve fitting was carried out using Equation 2:

$$v = \frac{V {max [ S ]}^{h}}{K h a l {f}^{h} + {[ S ]}^{h}}$$

### Generation of BfGAD Variants

The following B. fragilis glutamate decarboxylase mutants were created as shown in Table 2.

TABLE 2
B. fragilis Mutant Glutamate Decarboxylase DNA and Polypeptide Sequences

B. fragilis GAD_{D104N} Nucleotide Sequence (SEQ ID NO: 3)
GGTAGAGAAAATACCTGATGGTCCTACTACTCCGGAAATCGCCTATCAGATGGTGAAGGACGAAACTTTTGCTCAA
ACTCAGCCCCGTCTGAATCTTGCTACATGGGTGACTACTTATATGGATGATTATGCAACGAAGCTGATGAACGAAG
CTATCAATATCAACTACATTGATGAGACAGAATATCCTCGCATTGCTGTGATGAACGGTAAATGTATCAATATCGT
TGCTAATTTGTGGAACTCTCCGGAAAAAGATACCTGGAAAACCGGTGCATTGGCTATCGGTTCTTCAGAAGCTTGT
ATGTTGGGTGGTGTAGCTGCCTGGTTGCGTTGGCGCAAAAAACGTCAGGCCCAGGGTAAACCATTTGATAAACCTA
ACTTTGTCATTTCAACCGGTTTCCAGGTTGTTTGGGAAAAATTTGCTCAGTTGTGGCAGATTGAGATGCGTCAGGT
GCCTTTGACGCTGGACAAGACCACACTTGACCCGGAAGAAGCCCTGAAGATGTGTGATGAAAATACAATCTGTGTA
GTTCCAATCCAAGGGGTTACATGGACCGGTCTGAACGATGATGTTGAAGCCCTTGACAAAGCCCTCGATGCGTATA
ACGCTAAAACCGGTTATGATATTCCTATTCACGTAGACGCTGCCAGCGGTGGTTTCATCCTGCCGTTCCTGTATCC
TGATACCAAATGGGACTTCCGTCTGAAATGGGTTCTTTCCATCAGTGTATCCGGCCATAAGTTCGGTCTCGTATAT
CCGGGTTTGGGTTGGGTTGTCTGGAAAGGCAAAGAATATCTGCCCGAAGAAATGGCTTTCAGCGTAAACTACTTAG
GAGCTAACATTACTCAGGTAGGTTTGAACTTCTCTCGTCCTGCAGCTCAGATTTTGGGACAATATTATCAATTTAT
TCGTTTAGGATTCCAGGGATACAAGGAAGTACAATACAACTCTTTGCAGATTGCCAAATACATCCACAGCCAGATT
GCTAAGATGACTCCGTTCGTCAACTACTCGGAAGATGTAGTAAACCCGTTGTTCATTTGGTACATGAAGCCGGAAT
ATGCAAAGAACGCCAAATGGACTCTTTACGATTTGCAGGATAAGCTGGCTCAGCATGGCTGGATGGTTCCGGCATA
TACATTGCCTGCCAAGCTGCAAGATTATGTGGTTATGCGTGTCGTTGTCCGTCAGGGATTCAGTCGTGATATGGCC
GACATGTTGCTGGGCGACATTAAGAATGCTATTGCCGAACTGGAAAAACTGGAATACCCGACATCCACTCGTATTG
CCCAGGAGAAGAATCTGCCGGTAGAAGCCAAAGTATTTAACCATACCGGTAAACCACAAGCTGCCAAGAAATAA
B. fragilis GAD_{D104N} Peptide Sequence (SEQ ID NO: 4)
MEDLNFRKGDAKTEAFGSNRMLQPSPVEKIPDGPTTPEIAYQMVKDETFAQTQPRLNLATFVTTYMDDYATKLMNE
AININYINETEYPRIAVMNGKCINIVANLWNSPEKDTWKTGALAIGSSEACMLGGVAAWLRWRKKRQAQGKPFDKP
NFVISTGFQVVWEKFAQLWQIEMRQVPLTLDKTTLDPEEALKMCDENTICVVPIQGVTWTGLNDDVEALDKALDAY
NAKTGYDIPIHVDAASGGFILPFLYPDTKWDFRLKWVLSISVSGHKFGLVYPGLGWVVWKGKEYLPEEMAFSVNYL
GANITQVGLNFSRPAAQILGQYYQFIRLGFQGYKEVQYNSLQIAKYIHSQIAKMTPFVNYSEDVVNPLFIWYMKPE
YAKNAKWTLYDLQDKLAQHGWMVPAYTLPAKLQDYVVMRVVVRQGFSRDMADMLLGDIKNAIAELEKLEYPTSTRI
AQEKNLPVEAKVFNHTGKPQAAKK
B. fragilis GAD_{F81W} Nucleotide Sequence (SEQ ID NO: 5)
ATGGAAGATTTAAATTTCAGAAAAGGTGATGCTAAAACAGAAGCATTTGGTTCAAACAGAATGTTACAACCCTCTC
CGGTAGAGAAAATACCTGATGGTCCTACTACTCCGGAAATCGCCTATCAGATGGTGAAGGACGAAACTTTTGCTCA
AACTCAGCCCCGTCTGAATCTTGCTACATTCGTGACTACTTATATGGATGATTATGCAACGAAGCTGATGAACGAA
GCTATCAATATCAACTACATTAATGAGACAGAATATCCTCGCATTGCTGTGATGAACGGTAAATGTATCAATATCG
TTGCTAATTTGTGGAACTCTCCGGAAAAAGATACCTGGAAAACCGGTGCATTGGCTATCGGTTCTTCAGAAGCTTG
TATGTTGGGTGGTGTAGCTGCCTGGTTGCGTTGGCGCAAAAAACGTCAGGCCCAGGGTAAACCATTTGATAAACCT
AACTTTGTCATTTCAACCGGTTTCCAGGTTGTTTGGGAAAAATTTGCTCAGTTGTGGCAGATTGAGATGCGTCAGG
TGCCTTTGACGCTGGACAAGACCACACTTGACCCGGAAGAAGCCCTGAAGATGTGTGATGAAAATACAATCTGTGT
AGTTCCAATCCAAGGGGTTACATGGACCGGTCTGAACGATGATGTTGAAGCCCTTGACAAAGCCCTCGATGCGTAT
AACGCTAAAACCGGTTATGATATTCCTATTCACGTAGACGCTGCCAGCGGTGGTTTCATCCTGCCGTTCCTGTATC
CTGATACCAAATGGGACTTCCGTCTGAAATGGGTTCTTTCCATCAGTGTATCCGGCCATAAGTTCGGTCTCGTATA
TCCGGGTTTGGGTTGGGTTGTCTGGAAAGGCAAAGAATATCTGCCCGAAGAAATGGCTTTCAGCGTAAACTACTTA
GGAGCTAACATTACTCAGGTAGGTTTGAACTTCTCTCGTCCTGCAGCTCAGATTTTGGGACAATATTATCAATTTA
TTCGTTTAGGATTCCAGGGATACAAGGAAGTACAATACAACTCTTTGCAGATTGCCAAATACATCCACAGCCAGAT
TGCTAAGATGACTCCGTTCGTCAACTACTCGGAAGATGTAGTAAACCCGTTGTTCATTTGGTACATGAAGCCGGAA
TATGCAAAGAACGCCAAATGGACTCTTTACGATTTGCAGGATAAGCTGGCTCAGCATGGCTGGATGGTTCCGGCAT
ATACATTGCCTGCCAAGCTGCAAGATTATGTGGTTATGCGTGTCGTTGTCCGTCAGGGATTCAGTCGTGATATGGC
CGACATGTTGCTGGGCGACATTAAGAATGCTATTGCCGAACTGGAAAAACTGGAATACCCGACATCCACTCGTATT
GCCCAGGAGAAGAATCTGCCGGTAGAAGCCAAAGTATTTAACCATACCGGTAAACCACAAGCTGCCAAGAAATAA
B. fragilis GAD_{F81W} Peptide Sequence (SEQ ID NO: 6)
MEDLNFRKGDAKTEAFGSNRMLQPSPVEKIPDGPTTPEIAYQMVKDETFAQTQPRLNLATWVTTYMDDYATKLMNE
AININYIDETEYPRIAVMNGKCINIVANLWNSPEKDTWKTGALAIGSSEACMLGGVAAWLRWRKKRQAQGKPFDKP
NFVISTGFQVVWEKFAQLWQIEMRQVPLTLDKTTLDPEEALKMCDENTICVVPIQGVTWTGLNDDVEALDKALDAY
NAKTGYDIPIHVDAASGGFILPFLYPDTKWDFRLKWVLSISVSGHKFGLVYPGLGWVVWKGKEYLPEEMAFSVNYL
GANITQVGLNFSRPAAQILGQYYQFIRLGFQGYKEVQYNSLQIAKYIHSQIAKMTPFVNYSEDVVNPLFIWYMKPE
YAKNAKWTLYDLQDKLAQHGWMVPAYTLPAKLQDYVVMRVVVRQGFSRDMADMLLGDIKNAIAELEKLEYPTSTRI
AQEKNLPVEAKVFNHTGKPQAAKK
Mutant nucleotide codons or amino acids residues are bolded
D104N corresponds to amino acid D84 in SEQ ID NO: 2 or N84 in SEQ ID NO: 3
F81W corresponds to amino acid F61 in SEQ ID NO: 2 or W61 in SEQ ID NO: 6

The pET28a-BfGAD was used as a template for the generation of BfGAD variants, BfGAD_{D104N} and BfGAD_{F81W}, by site-directed mutagenesis with Phusion DNA polymerase using the following primer pairs incorporating codons for the specific amino acid substitution (Table 3, underlined):

TABLE 3
Primers for Site Directed Mutagenesis (5′→3′)
Primers for BfGAD_{D104N}

| | | |
|---|---|---|
| BfGAD_{D104N} Fwd | CCGCAATGCGCGGATATTCGGTTTCATTAATATAGTTAATG | SEQ ID NO: 7 |
| BfGAD_{D104N} Rev | CATTAACTATATTAATGAAACCGAATATCCGCGCATTGCGG | SEQ ID NO: 8 |

Primers for BfGAD_{F81W}

| | | |
|---|---|---|
| BfGAD_{F81W} Fwd | CATCCATATAGGTGGTCACCCAGGTCGCCAGGTTCAGGCGCG | SEQ ID NO: 9 |
| BfGAD_{F81W} Rev | CGCGCCTGAACCTGGCGACCTGGGTGACCACCTATATGGATG | SEQ ID NO: 10 |

D104 corresponds to amino acid 84 in SEQ ID NO: 2
F81 corresponds to amino acid 61 in SEQ ID NO: 2

The variants were confirmed by gene sequencing and full plasmid sequencing (Plasmidsaurus).

Continuous Activity Assays with D-Glutamate

The activity assays of BfGAD (WT and variants) were conducted by performing GABase assay with 50 mM D-glutamate as a substrate as mentioned above.

### CO₂ (g) Detection by Headspace Gas Chromatography

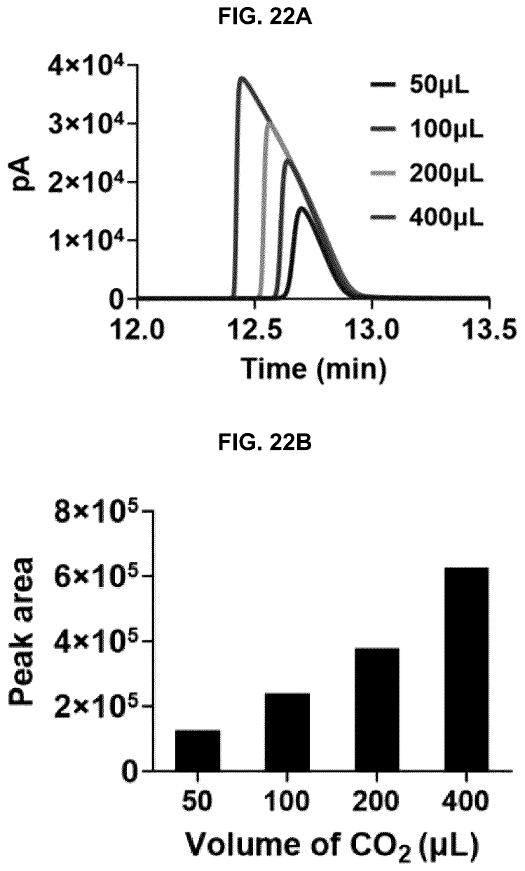

All enzymatic reactions and controls were prepared in 6 ml vials and sealed prior to starting the reaction. The reactions were started by the addition of BfGAD (WT or variants) with a syringe into the reaction mixture containing 50 mM substrate in 50 mM sodium acetate buffer, pH 4.7. These vials were incubated at 37° C. for 24 hours and 48 hours. Headspace GC measurements were carried out on an Agilent 8890 gas chromatograph system equipped with a flame ionization detector (FID) and a Hayesep Q packed column (1.8 m×2 mm×3.17 mm) (Agilent) operating with an Argon carrier gas (flow rate=5 mL/min). The oven was programmed to hold 30° C. for 6.5 min, ramp at 30° C./min to 280° C. with a hold for 4 min for a total run time of 18.83 min. The flame ionization detector was used for the detection of CO₂ gas with a temperature setting of 275° C., hydrogen flow of 60 mL/min, air flow of 400 mL/min and constant makeup gas (nitrogen) at 5 mL/min. The retention time for CO₂ gas was 12.83 min. Multiple standards of CO₂ gas were analyzed by this method to confirm retention time before injecting reaction samples (FIG. 22A-B).

### Thin Layer Chromatography (TLC)

Silica gel plates (stationary phase) with a solvent system (mobile phase) of 3:1:1 ratio of butanol:acetic acid:H₂O were used to separate reaction products from substrates. 2 μL of reaction mixtures were spotted on glass silica plates spotted with 10 mM of various metabolites (L-glutamate, D-glutamate, L-CSA, L-CA, L-HCA, L-Aspartate, GABA, hypotaurine, taurine, homotaurine and β-alanine) as standards. Separation via TLC was carried out at 25° C. for 3-4 hours in an above-mentioned mobile phase. Once mobile phase reached a sufficient height on the TLC plate, chromatographic separation was discontinued. Plates were treated with 0.5% ninhydrin in acetone (w/v) and heated minimally with a dryer for the color development. The TLC plate images were initially inverted via ImageJ and then intensity of each spot was quantified by a custom python code.

Confirmation of Alternate Products of BfGAD (WT or Variants) Reactions by Liquid Chromatography with Tandem Mass Spectrometry (LC-MS/MS)

Samples were analyzed by ESI-LC-MS/MS in positive (products) and negative ion (substrates) mode using a Thermo vanquish LC and TSQ Altis Triple Quadrupole Mass Spectrometer. Samples were separated by gradient elution using Agilent Infinity lab Poroshell 120 HILIC-Z 2.1×100 mm 2.7 μm column at 25° C. with 20 mM ammonium formate, pH 3.0 (Solvent A) and 20 mM ammonium formate in 9:1 acetonitrile: H₂O, pH 3.0 (Solvent B). Elution was initiated at 70% B for 11.5 min, followed by gradient elution from 70 to 100% B over 11.5 min, 100% B for 4 min at a flow rate of 0.5 mL/min. The precursor to product transitions of m/z [M+H]⁻ 152→88.1 (Cysteine sulfinic acid), [M+H]⁻ 168→81.1 (Cysteic acid), [M+H]⁺ 110.1→45.1 (hypotaurine), [M+H]⁺126.1→44.1 (taurine) were employed to monitor substrates and products.

### Multiple Sequence Alignment and Phylogenetic Analyses of Gut Microbial GADs

All the protein sequences were obtained from NCBI. Multiple sequence alignment was performed using Clustal Omega (1.2.4) tool with default parameters to understand sequence similarities among various gut microbial glutamate decarboxylases. The phylogenetic tree was created using cyberinfrastructure for phylogenetic research (CIPRES). Within CIPRES, the MAFFT on XSEDE (7.505) was utilized to create a separate multiple sequence alignment that goes through FastTreeMP on XSEDE (2.1.10) to obtain the phylogenetic tree file which can be visualized and annotated with Interactive tree of life (iTOL v6.8.1).

BfGAD Dimer Generation with the AlphaFold and Structural Alignment Parameters Via PyMol

Dimer structure of wild type and variants of BfGAD were generated with AlphaFold2 with MMseqs2 (ColaBfold v1.5.3). All structural alignments were carried out using PyMOL align method that uses five iteration cycles and a cutoff of 2 Å.

### Glutamate Decarboxylase (GAD) are Prevalent in the Bacteroides Genus

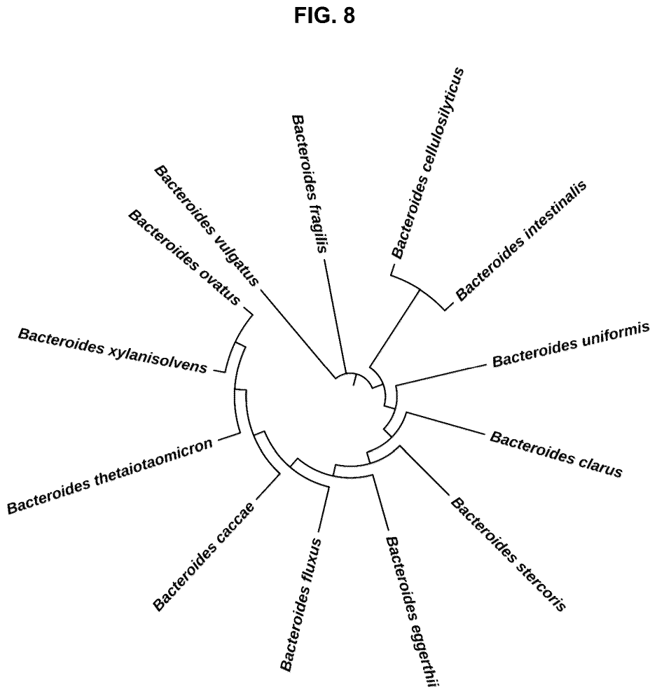

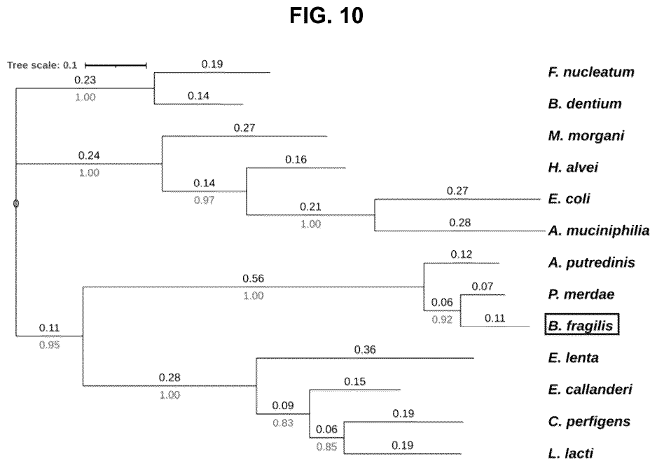

A bioinformatics analysis revealed that many species of the Bacteroides genus harbor genes annotated as GAD (FIG. 8). Here, the focus was on a GAD (BfGAD) from the model gut microbe, Bacteroides fragilis. A multiple sequence alignment was performed with gut microbial GADs including BfGAD, which showed that several catalytic residues predicted to be involved in substrate binding or cofactor (PLP) binding are highly conserved (FIG. 9). Additionally, a phylogenetic analysis of BfGAD with other gut microbial GADs was performed. In a maximum likelihood phylogenetic tree, it was evident and observed that the annotated glutamate decarboxylases from Alistipes putredinis and Parabacteroides merdae are closely related to BfGAD (FIG. 10).

### Wild-Type BfGAD (BfGAD_{WT}) Forms an Oligomer

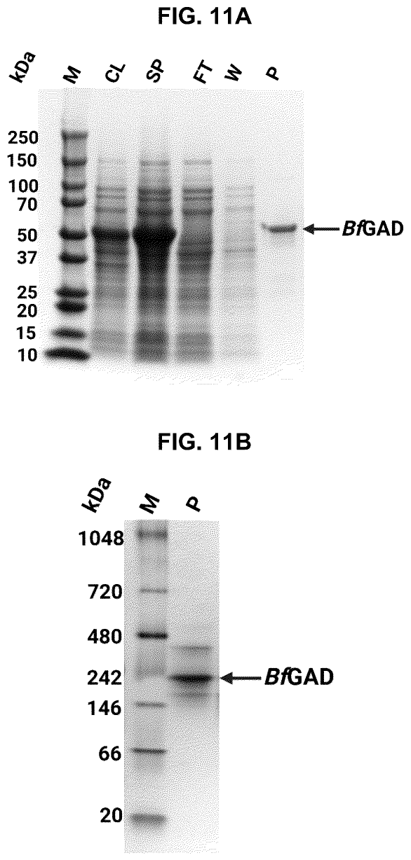

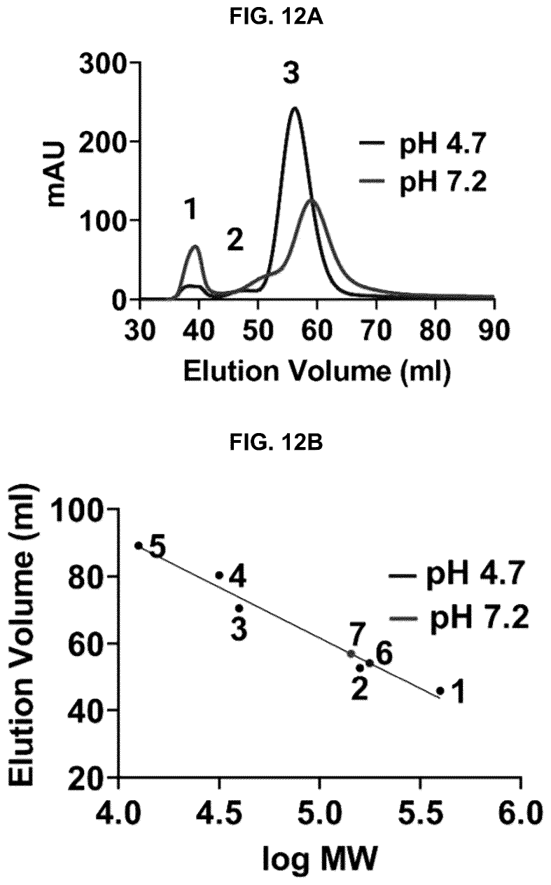

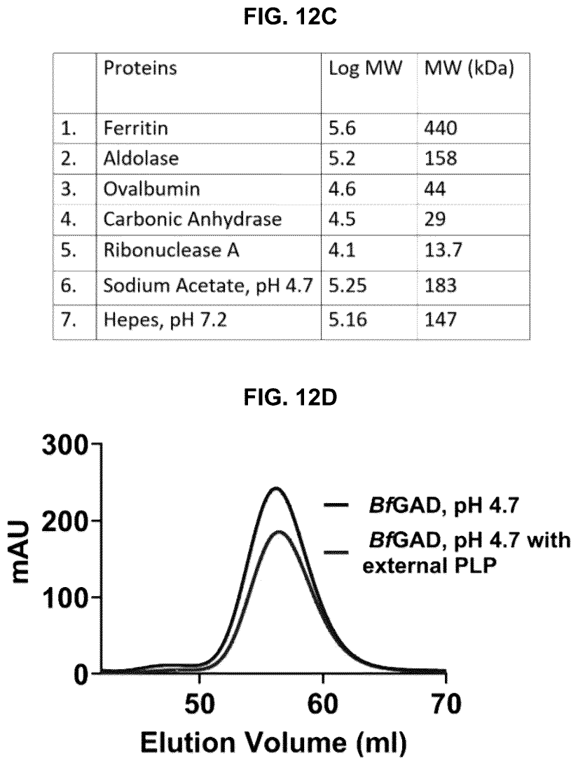

The recombinant wild-type BfGAD (BfGAD_{WT}) with an N-terminal His-tag was purified by Ni-NTA affinity chromatography. A protein band with an approximate expected mass of a BfGAD_{WT} monomer (calculated mass 56 kDa) was observed with SDS PAGE, while a band indicating a tetrameric oligomer was observed with a native PAGE (FIG. 11A-B). Oligomeric composition of the BfGAD_{WT}, as determined by gel filtration chromatography, is presented as chromatograms for the BfGAD_{WT} at an acidic pH and a neutral pH (FIG. 12A). Peak 1 is the void volume of the column indicating protein aggregates. There are two peaks representing two different oligomeric compositions (2 and 3) observed at each pH. Peak 2 is broad and corresponds to approximately 351 kDa, indicating a hexamer. Peak 3 is the major peak and shows an approximate molecular weight that is higher than the dimer but lower than a tetramer at pH 4.7, whereas at pH 7.2 the peak suggests a mass that is closer to a dimer than a tetramer (FIG. 12A-C, 180 kDa at pH 4.7 and 150 kDa at pH 7.2) for BfGAD_{WT} (FIG. 12A-C). Furthermore, the addition of external PLP did not change the oligomeric state of the BfGAD_{WT} (FIG. 12D). Different oligomeric compositions of microbial GADs have been reported so far. These data suggest that BfGAD_{WT} might have dimeric oligomeric composition.

### PLP Cofactor Bound to the BfGAD_{WT} Undergoes pH Dependent Tautomeric Changes

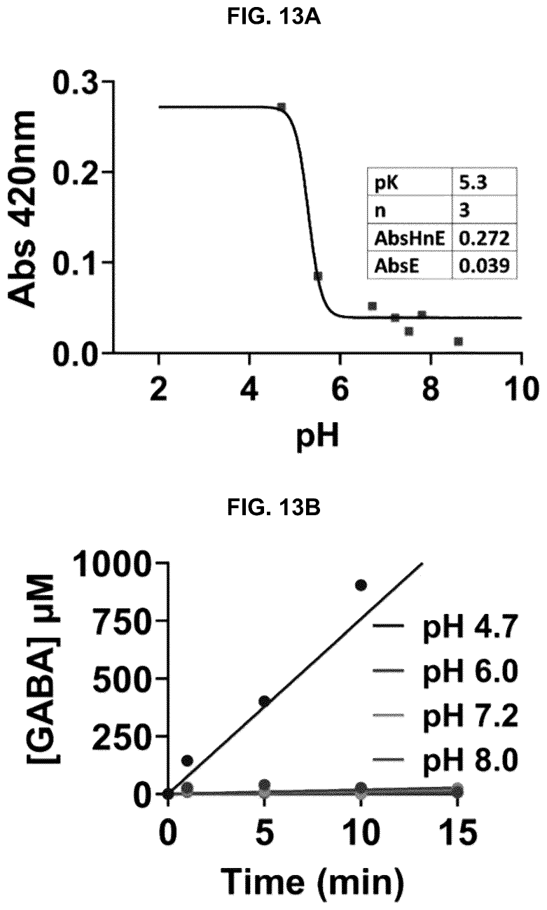

Spectral studies with BfGAD_{WT} revealed that the enzyme was purified in its holo form with the covalently bound PLP cofactor. There were two absorption maxima observed for the PLP bound BfGAD_{WT}—335 nm and 420 nm (FIG. 1B). These absorption maxima result from two tautomeric forms of the enzyme bound PLP cofactor, enolimine form at 335 nm and ketoenamine form at 420 nm. The proportions of these tautomers vary depending on the pH (FIG. 1B). At lower pH, the species absorbing at 420 nm was more dominant, whereas at higher pH, 340 nm absorbing form became more prevalent. With increase in pH from 4.7 to 8.6, there was a decrease in 420 nm absorbing species with concomitant increase in 340 nm absorbing species (FIG. 1B). These changes in the absorbances of the bound PLP cofactor as the function of the change in pH with BfGAD_{WT}, have been seen with other microbial GADs. The analysis of the absorbance versus pH curve for the 420 nm wavelength indicated that the transition in absorbance in response to pH variation is influenced by the involvement of multiple protons (FIG. 13A). Previously with E. coli GAD, the less hydrated active site at neutral pH drove the formation of enolimine tautomer (340 nm) whereas more polar active site at acidic pH predominantly produced ketoenamine tautomer (420 nm). Additionally, the alterations in cofactor absorbance corresponding to different pH were in close agreement with the activity profiles of BfGAD_{WT} measured across the same range of pH values. Particularly, at pH 4.7, where the enzyme bound PLP predominantly absorbed at 420 nm, the BfGAD_{WT} also exhibited its maximum catalytic activity (FIG. 13B). Additionally, enzymatic activity was found to be minimal at pH other than 4.7 (FIG. 13B). Therefore, all subsequent activity assays were carried out at pH 4.7.

### Evidence for Allosteric Regulation of BfGAD_{WT}

The activity assays conducted with varying BfGAD_{WT} concentrations exhibited a linear relationship between the initial reaction velocities and enzyme concentrations (FIG. 1C). From the plot of enzyme concentration variation, an optimal BfGAD_{WT} concentration was found for subsequent kinetic assays. Interestingly, while varying L-glutamate concentrations, the plot of initial velocity versus substrate concentration displayed a sigmoidal curve instead of a typical hyperbolic dependence, suggestive of an allostery in BfGAD_{WT}. From the fit of this plot with the hill equation (Eq. 2), the analyzed K_{0.5} (K_{half}) was 8.6±0.4 mM and Vₘₐₓ was 0.86±0.04 mM/min with a hill coefficient of 1.7 (FIG. 1D).

### Evolved BfGADs Exhibit Catalytic and Structural Perturbations

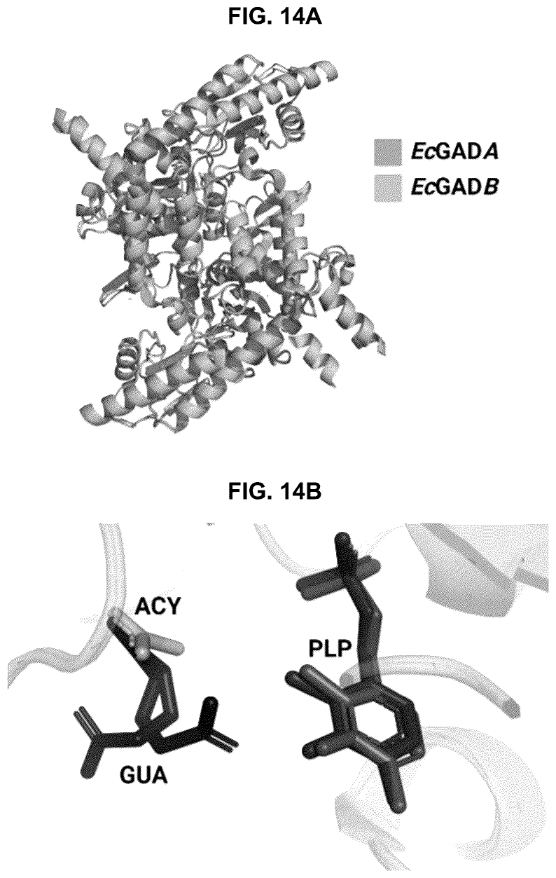

The earliest microbial GAD structures were solved for E. coli GADs (GADA, PDB ID 1XEY and GADB, PDB ID 1PMM) (FIG. 14A-B). Both structures were solved with the bound ligand in the active site. Based on the interactions of the acetate ion in the GADB active site and glutarate (substrate analog) in the GADA active site (FIG. 14B), residues for the evolution of the BfGAD_{WT} were selected via rational design. Two such residues that are present in the active sites of both EcGADs are phenylalanine 63 (corresponds to F81 in BfGAD_{WT}) and aspartate 86 (corresponds to D104 in BfGAD_{WT}) that make H-bonding interactions with one of the carboxylates of the substrate analog and an acetate ion. Specifically, in E. coli GADB ligand bound structure, the carboxylate group of acetate received a hydrogen bond from the amide nitrogen of the F63 (F81 BfGAD_{WT}) and a side chain carboxylate of D86 (D104 BfGAD_{WT}) of the neighboring subunit. Whereas in E. coli GADA ligand bound structure, one of the carboxylate groups of the glutarate (substrate analogue) forms H-bond with the amide nitrogen of F63 (F81 BfGAD_{WT}) and with the side chain carboxylate of D86 (D104 BfGAD_{WT}) of the neighboring subunit. These residues are completely conserved in annotated GADs from prominent gut microbes with the exception of Eggerthella lenta that harbors shorter GAD with the absence of a conserved phenylalanine (FIG. 9). Most microbial GADs are functionally active as dimers, with residues from both monomers contributing to the active site.

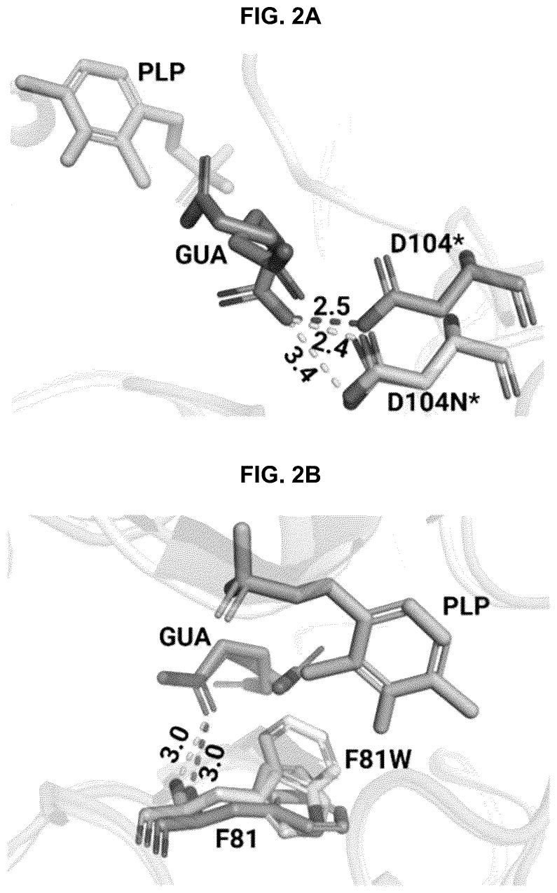

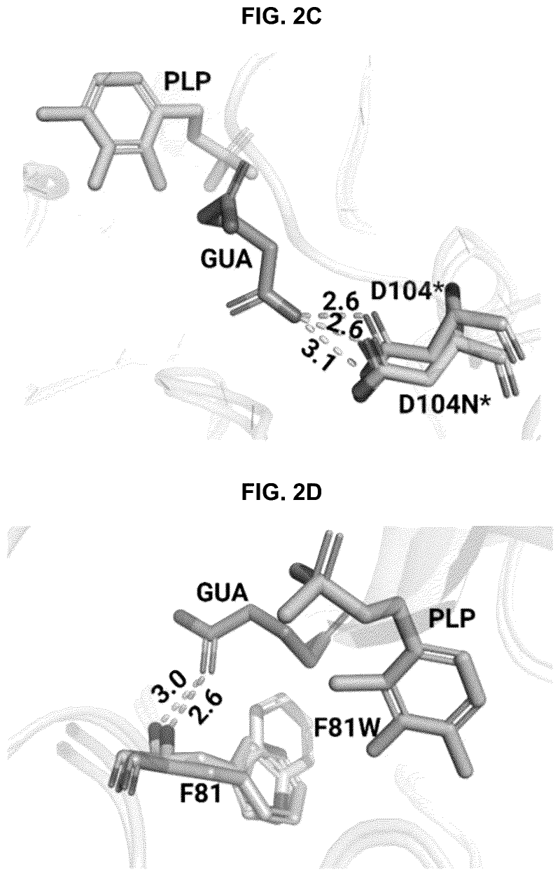

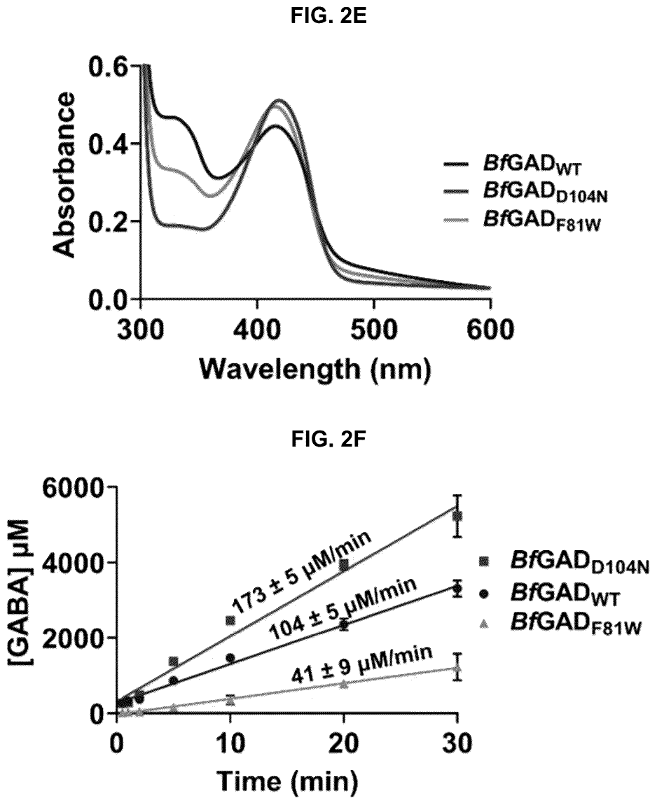

Recently, structure of glutamate decarboxylase from Bacteroides thetaiotaomicron (BtGAD), bound with a substrate analog (glutarate) was solved. With these available structures, the substrate analog (glutarate) bound structures of EcGADA (PDB ID 1XEY) and BfGAD (PDB ID 7X51) were aligned with BfGAD_{WT} dimer created with AlphaFold (FIG. 2). Based on these structural alignments, two enzymes were engineered by introducing single amino acid alterations at two separate positions to test if the substrate preference of the BfGAD_{WT} could be evolved. These changes were Asp104Asn (D104N) and Phe81Trp (F81W) (FIG. 9). FIG. 2A-B illustrate the locations of these residues within the active site of BfGAD, in close proximity to the bound glutarate and the cofactor PLP, with the active site of EcGADA superimposed on the BfGAD dimer. Similarly, FIG. 2C-D display aligned BfGAD and BtGAD structures with glutarate and PLP bound in the active stie of BfGAD. AlphaFold generated models of the engineered enzymes, BfGAD_{D104N} (FIGS. 2A and C) and BfGAD_{F81W} (FIGS. 2B and D) were then superimposed with ligand bound structures of EcGAD and BtGAD. The rotamers for the altered residues were selected based on their best fit to the position of wild-type residues in the protein BfGAD_{WT}.

Both of these variants, BfGAD_{D104N} and BfGAD_{F81W}, were able to be purified with the same conditions as the BfGAD_{WT}. Both variants were active and were purified as holoenzymes (PLP bound form). Additionally, UV-Vis spectra of BfGAD_{WT} and both variants were collected at pH 4.7 (FIG. 14C). Although all three enzyme preparations showed absorbance maxima at both 335 nm and 420 nm, the proportions of these two absorbing tautomeric species vary in all three enzymes. Additionally, initial velocity was measured for both variants in the presence of the native substrate L-glutamate. Progress curves for engineered BfGADs along with BfGAD_{WT}, conducted in the presence of saturating concentration of the native substrate (L-glutamate), showed variations in the initial velocities of decarboxylations between wildtype and the engineered enzymes (FIG. 14D). BfGAD_{D104N} showed a 1.7-fold increase whereas BfGAD_{F81W} showed 2.5-fold decrease in the initial velocity compared to the BfGAD_{WT}.

Wild-Type and Evolved BfGADs can Decarboxylate Substrates Other than L-Glutamate

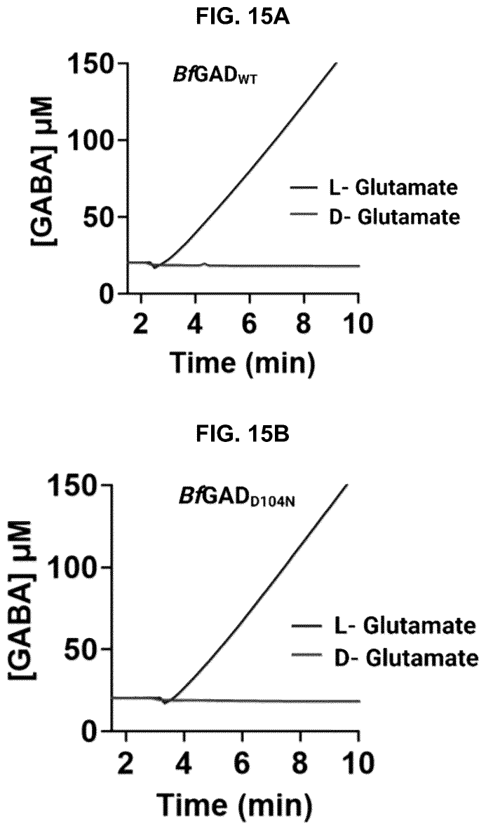

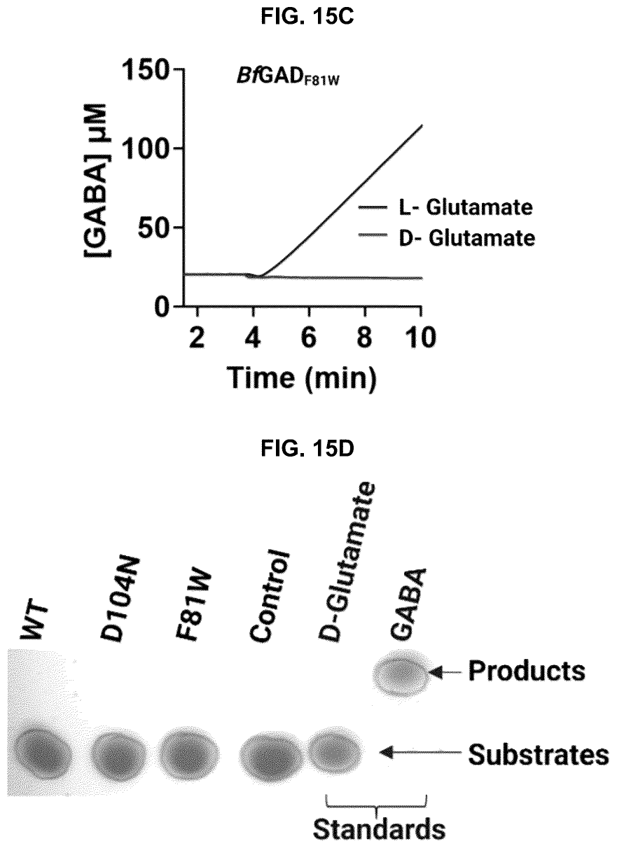

As mentioned above, the purified BfGAD_{WT} is able to catalyze the conversion of L-glutamate to GABA. To test if it could use the D-form of the glutamate as a substrate, catalytic assays with D-glutamate were conducted using the GABase assay system and TLC. The results showed that both the wild-type and engineered BfGADs were unable to utilize D-glutamate as a substrate (FIG. 15). Most microbial GADs studied so far show high substrate specificity towards L-glutamate. However, there are examples of archaeal GADs that prefer L-aspartate over L-glutamate. An archaeal GAD from P. horikoshii also shows decarboxylation activity with L-cysteate. In addition to archaeal GADs, certain mammalian glutamate decarboxylase (GAD) homologs are known to decarboxylate one or more of the non-native substrates including as L-aspartate, L-cysteate, and L-cysteine sulfinate. Moreover, proteins similar to GADs have been demonstrated to form taurine in some marine microbes. However, there are limited systematic studies investigating the production of taurine, its analogs, and β-alanine using L-CA (cysteate), L-CSA (cysteine sulfinate), L-HCA (homocysteate), and L-aspartate as substrates (Scheme 1), specifically for gut microbial GADs. Based on two key observations, it was hypothesized that gut microbial GADs might utilize these molecules as substrates: 1. E. coli GAD can accept the phosphonated form of L-glutamate as a substrate; and 2. Archaeal and eukaryotic GADs exhibit diverse substrate specificities, enabling them to catalyze the decarboxylation reactions of non-native substrates.

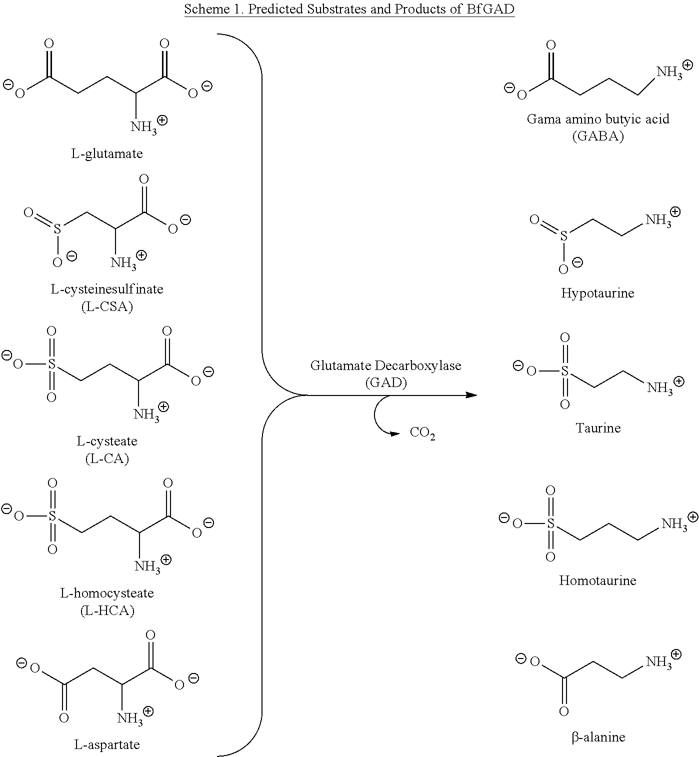

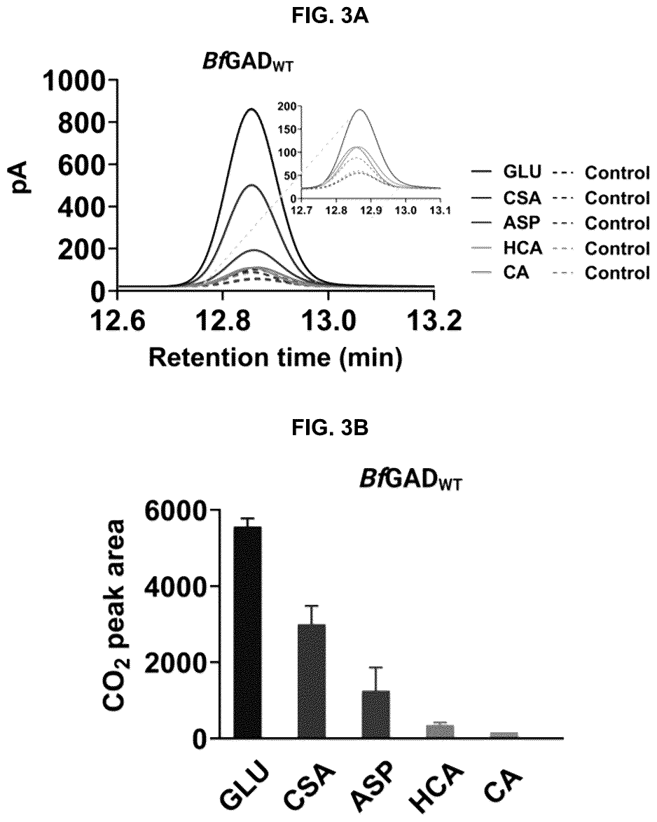

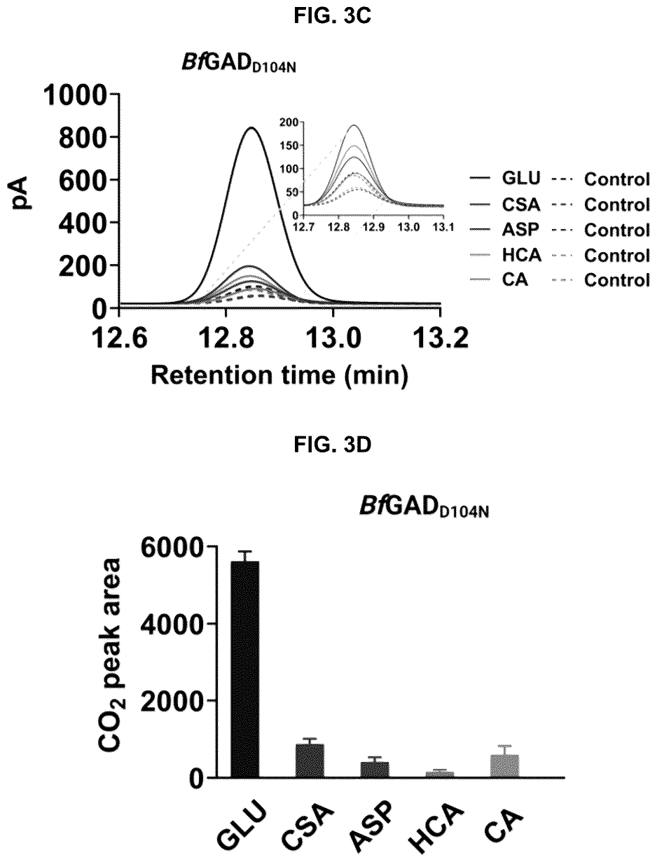

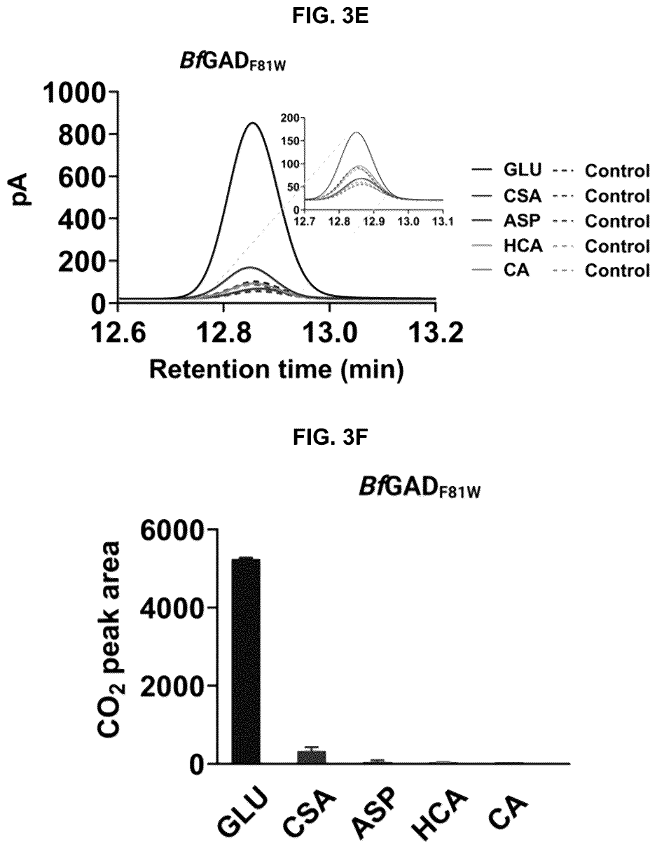

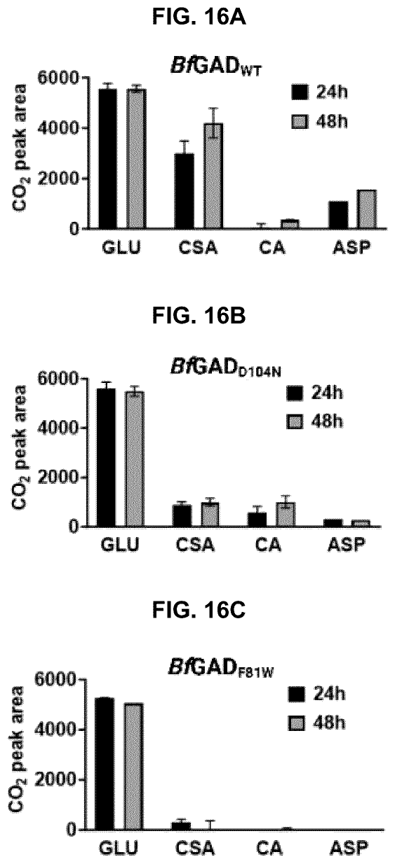

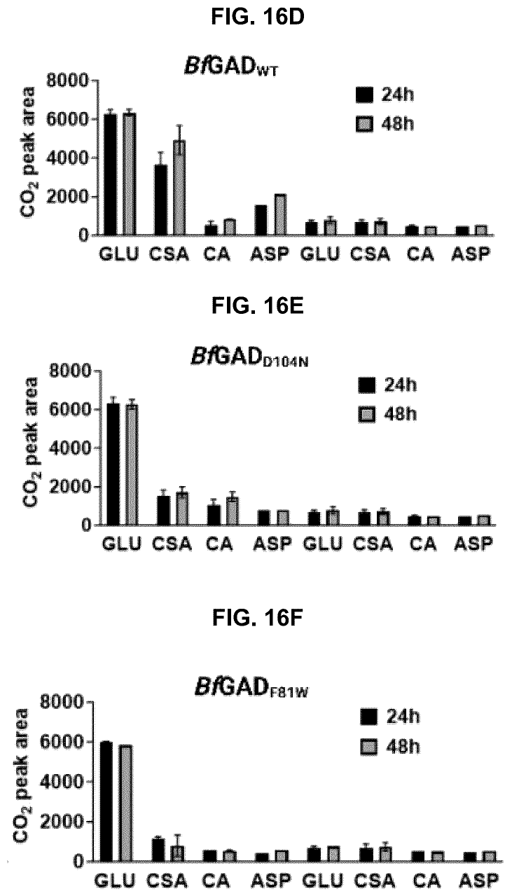

If BfGAD_{WT} or evolved BfGADs are able to decarboxylate substrates other than the native substrate L-glutamate, then the detection of the common product CO₂ will be a positive test for the utilization of other substrates (Scheme 1). For this reason, a headspace GC was used to measure the CO₂ evolved from reactions catalyzed by either BfGAD_{WT} or evolved BfGADs during the decarboxylation of various substrates. FIG. 3 shows data collected for BfGAD_{WT} and engineered BfGADs with five different substrates via headspace GC. FIGS. 3A, 3C, and 3E demonstrate chromatograms with the CO₂ peaks, visible immediately after 12.8 minutes produced by the reactions of BfGAD_{WT}, BfGAD_{D104N}, and BfGAD_{F81W} respectively with five different substrates. Whereas FIGS. 3B, 3D, and 3F depict CO₂ peak areas from the decarboxylation reactions of the substrates after 24 hours incubation with either BfGAD_{WT} or engineered BfGADs. Here, BfGAD_{WT} showed significant production of CO₂ generated from the decarboxylation of both L-glutamate and L-CSA (FIG. 3A-B). This is the first indication of a gut microbial GAD, specifically a GAD from Bacteroides, that can utilize L-CSA as a substrate. In addition to L-CSA, BfGAD_{WT} was capable of decarboxylating L-HCA and L-CA to small extents as evidenced by the small CO₂ peaks (FIG. 3A) in the chromatogram and small peak areas for evolved CO₂ (FIG. 3B). Additionally, BfGAD_{WT} was able to decarboxylate L-aspartate (FIG. 3A-B). For BfGAD_{WT}, if CO₂ production peak area with the native substrate L-glutamate is considered to be 100%, then 54%, 3%, 6% and 20% of CO₂ production was observed with L-CSA, L-CA, L-HCA, and L-Asp, respectively, within 24 hours, which increased to 75% (L-CSA), 6% (L-CA), 7% (L-HCA), and 28% (L-Asp) within 48 hours compared to the native substrate L-glutamate (FIG. 16A).

For BfGAD_{D104N}, CO₂ production was comparable to the BfGAD_{WT} while using native substrate L-glutamate. Compared to the peak area of CO₂ production for L-glutamate (native substrate), around 16%, 11%, 3%, and 7% CO₂ production was observed from L-CSA, L-CA, L-HCA, and L-aspartate respectively within 24 hours (FIG. 3C-D). These percentages increased to 18% for both L-CSA and L-CA, 7% for L-HCA and decreased to 4% with L-aspartate within 48 hours (FIG. 16B). Based on the data collected with BfGAD_{WT} and BfGAD_{D104N}, it was proposed that residue Asp104 (D104) from the neighboring monomer is playing an important role in accommodating various substrates. This engineered enzyme was also able to decarboxylate L-CSA, L-HCA, and L-Asp in addition to the native substrate L-glutamate but less efficiently than the BfGAD_{WT}, specifically the activity towards L-CSA was significantly impacted. It is possible and likely that the negative charge of the side chain carboxylate from D104 is important for the specificity towards alternative substrates L-CSA, L-HCA, and L-Asp but not for the native substrate L-glutamate. As a result, when the charge was removed due to the substitution of Asp (D) to Asn (N) at position 104, the catalysis with these alternate substrates was affected, but not with the native substrate L-glutamate. Interestingly, a 2-2.5-fold increase in the production of taurine was noticed with this evolved enzyme compared to the BfGAD_{WT} (FIG. 3D), indicating that the substitution from D to N was favorable for taurine production.

The second engineered enzyme, BfGAD_{F81W} retained its activity with the native substrate L-glutamate as well but as observed in the initial velocity experiments (FIG. 2F), it was slower compared to BfGAD_{WT}. If the CO₂ peak area of BfGAD_{WT} or BfGAD_{D104N} with the native substrate L-glutamate is considered to be 100%, BfGAD_{F81W} showed the peak area of around 93-94% with the same concentration of the substrate. Additionally, there was minimal decarboxylation activity of BfGAD_{F81W} with L-CA, L-HCA, and L-ASP. However, this evolved enzyme still retained considerable decarboxylation activity with L-CSA. It is interesting to note that a bulky substitution of tryptophan (W) at position 81, instead of phenylalanine (F) (FIG. 2B, 2D), made the enzyme more specific towards the native substrate L-glutamate. Based on the CO₂ evolution captured by the GC data, the substrate preference for BfGAD_{WT} was L-Glu>L-CSA>L-Asp>L-HCA>L-CA that changed to L-Glu>L-CA>L-CSA>L-Asp>L-HCA for BfGAD_{D104N}. Whereas for BfGAD_{F81W}, only L-glutamate and L-CSA showed decarboxylation and L-glutamate was a much better substrate than L-CSA.

### Wild-Type and Evolved BfGADs Produce Multiple Neuromodulatory Molecules

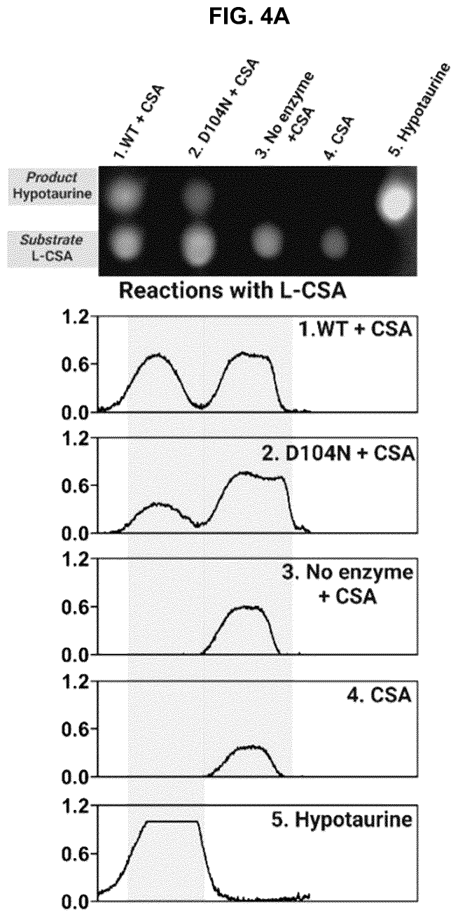

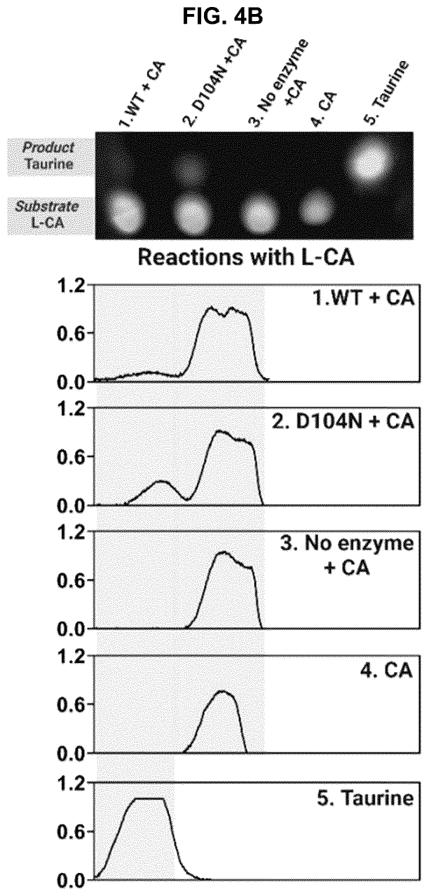

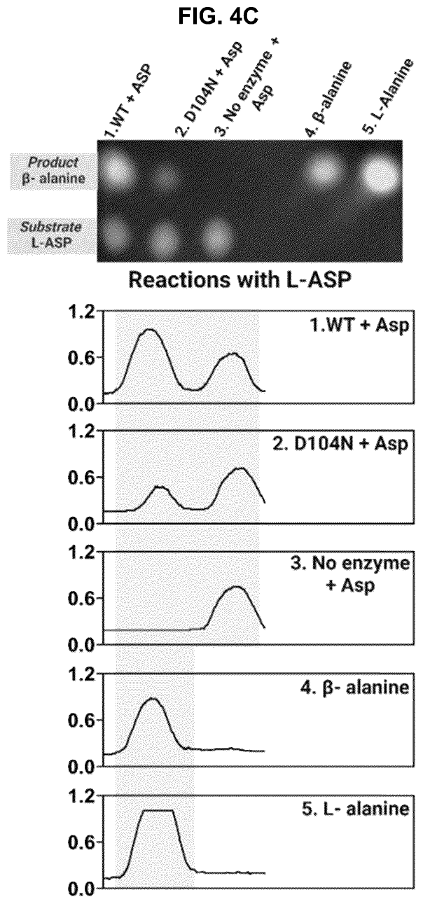

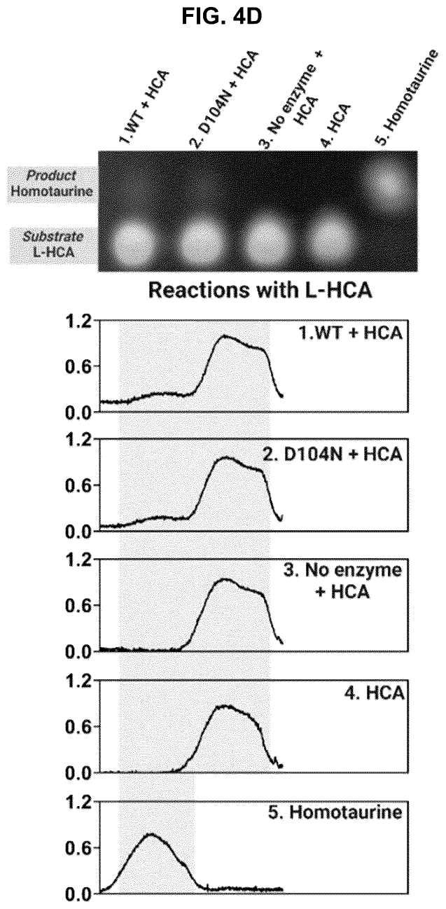

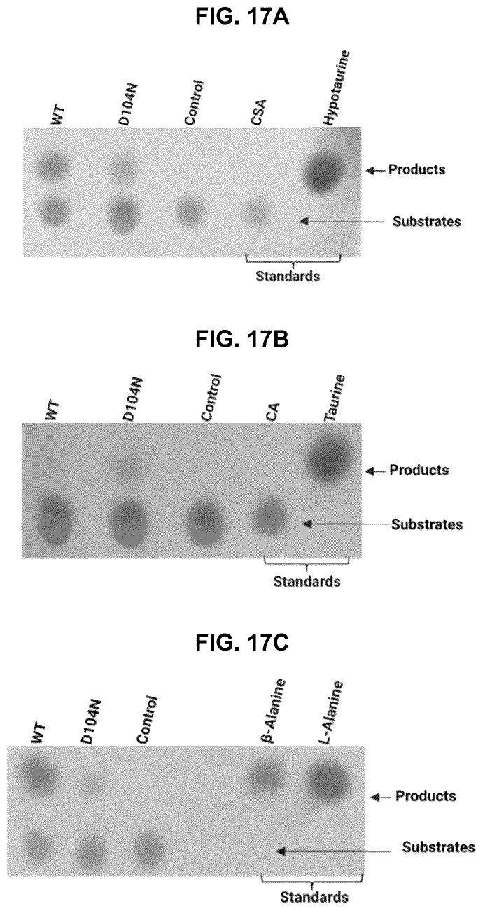

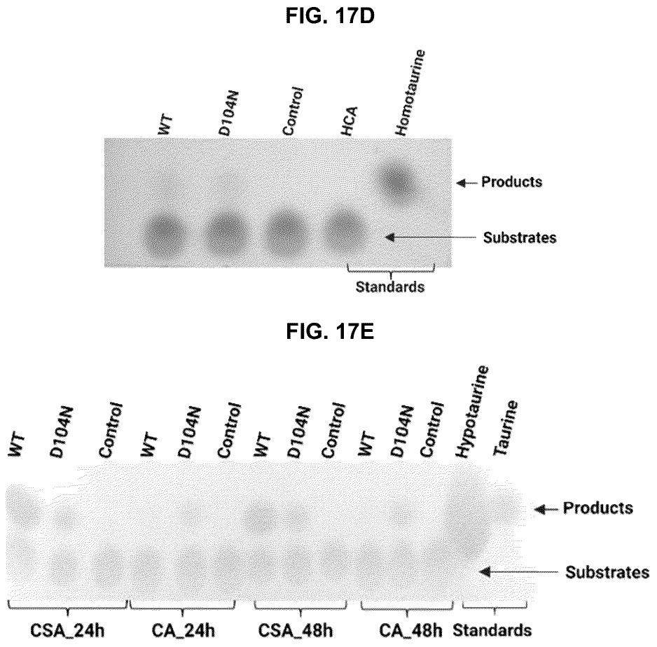

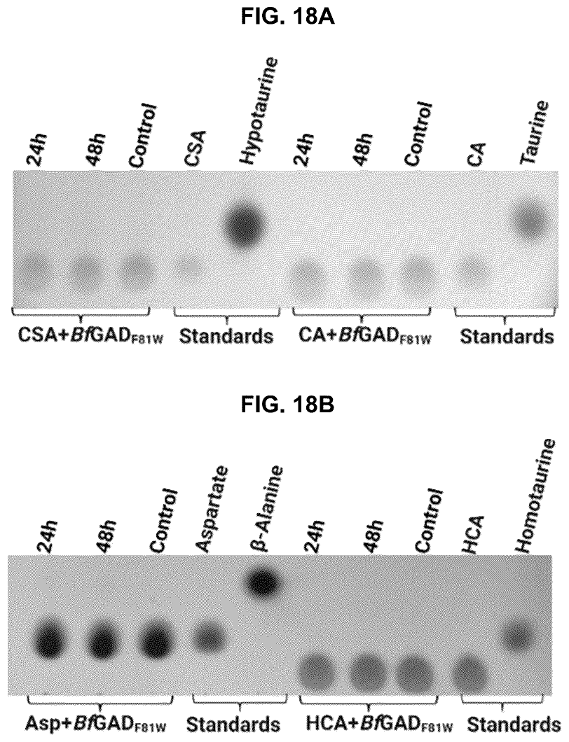

The generation of CO₂ in reactions facilitated by BfGAD_{WT} and its engineered variants allowed for the identification of products resulting from decarboxylation. Both BfGAD_{WT} and BfGAD_{D104N} produced decarboxylated products-hypotaurine, taurine, homotaurine, and β-alanine from substrates L-CSA, L-CA, L-HCA, and L-Asp respectively (FIG. 4A-D, FIG. 17A-D). Intensity plots generated from TLC plates showed that BfGAD_{WT} decarboxylated L-CSA and L-Asp to produce hypotaurine and β-alanine more efficiently than BfGAD_{D104N} (FIG. 4A, C). However, BfGAD_{D104N} was better at decarboxylating L-CA to taurine than BfGAD_{WT} (FIG. 4B). A small amount of decarboxylated product taurine with BfGAD_{WT} from L-CA decarboxylation was observed during a 24-hour incubation, whereas BfGAD_{D104N} showed a significant production of taurine during this time frame (FIG. 4B, FIG. 17E). While the CO₂ evolution experiments have shown that BfGAD_{WT} can catalyze L-CA decarboxylation in 24 hours, the failure to observe decarboxylated product on TLC may be attributed to the low concentration of taurine produced, making it undetectable on a TLC plate. Both enzymes BfGAD_{WT} and BfGAD_{D104N} were able to catalyze the decarboxylation of L-HCA to homotaurine to a very small extent (FIG. 4D). It was also verified that the product from the L-Asp decarboxylation was β-alanine and not L-alanine. Although, the Rf (retention factor) values were almost similar for β-alanine and L-alanine, the staining with the ninhydrin differs for these molecules. β-alanine exhibited a purple color whereas L-alanine shows a brick red color with ninhydrin stain (FIG. 17C). No activity of BfGAD_{F81W} with L-CA, L-Asp, and L-HCA was found and no taurine, β-alanine, and homotaurine were detected even after 48-hour incubation (FIG. 18A-B). These results reinforce the observations from CO₂ evolution experiments. Additionally, despite the detection of CO₂ during the decarboxylation activity of BfGAD_{F81W} with L-CSA (FIG. 3F), no detectable hypotaurine spots were observed on the TLC plate (FIG. 18A). It was hypothesized that the amount of hypotaurine produced by BfGAD_{F81W} may not be sufficient to be detected via TLC, as a higher concentration of molecules (in mM range) are needed for TLC analysis.

BfGAD_{WT} and BfGAD_{D104N} Generate Neuromodulatory Molecules at Differential Abundances, Even when Presented with a Mixed Substrate Pool

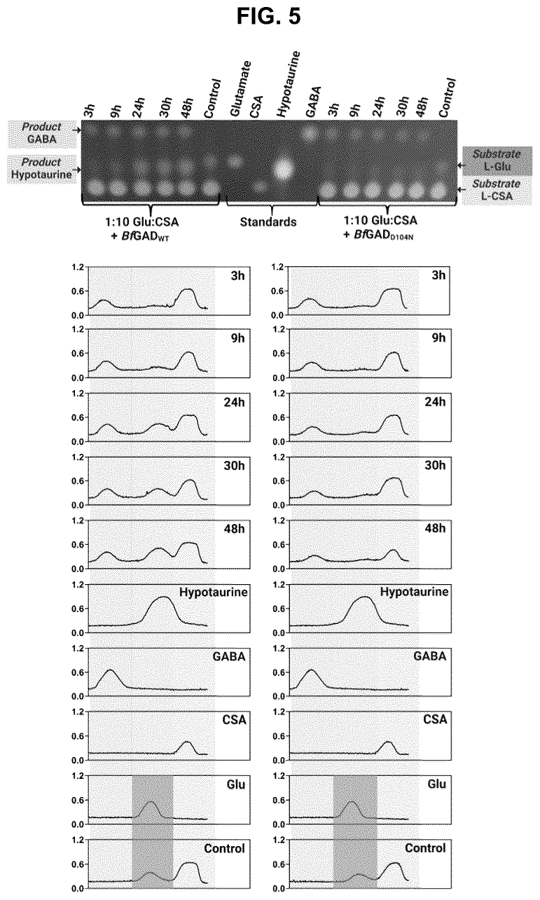

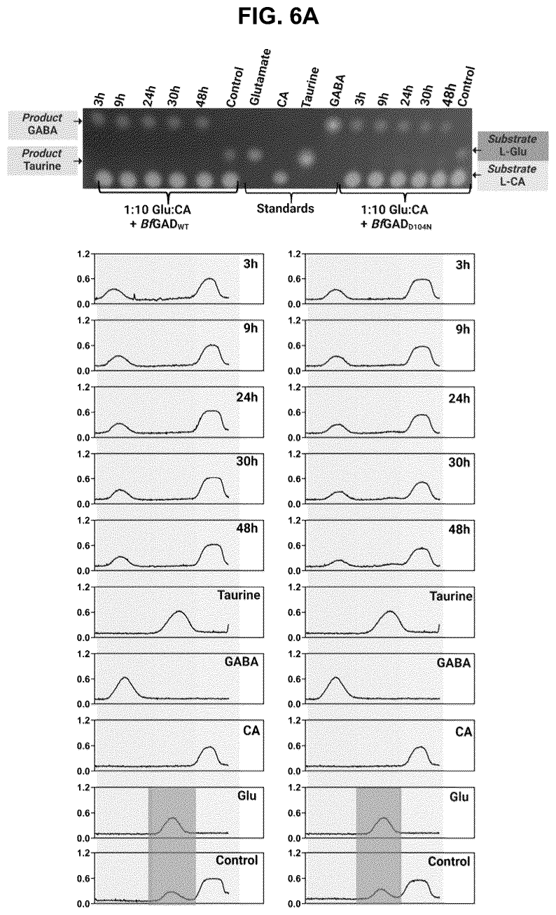

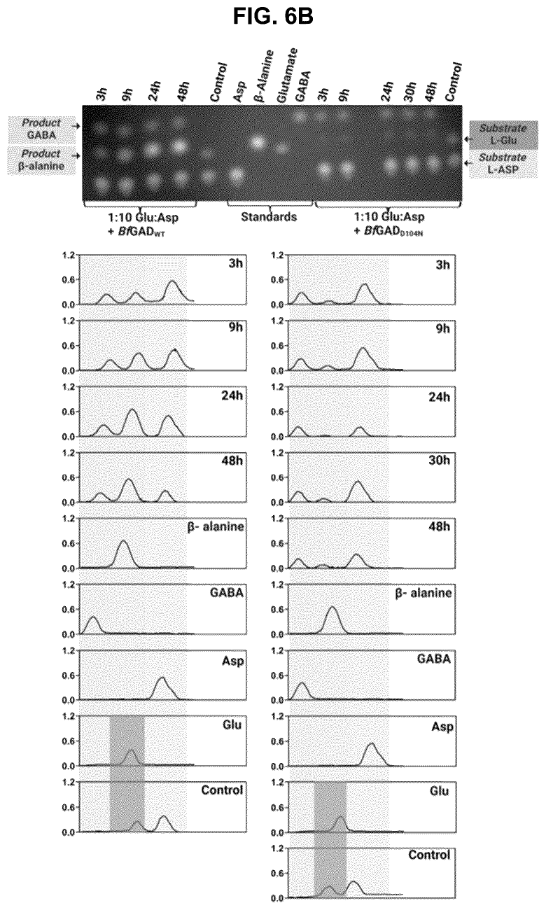

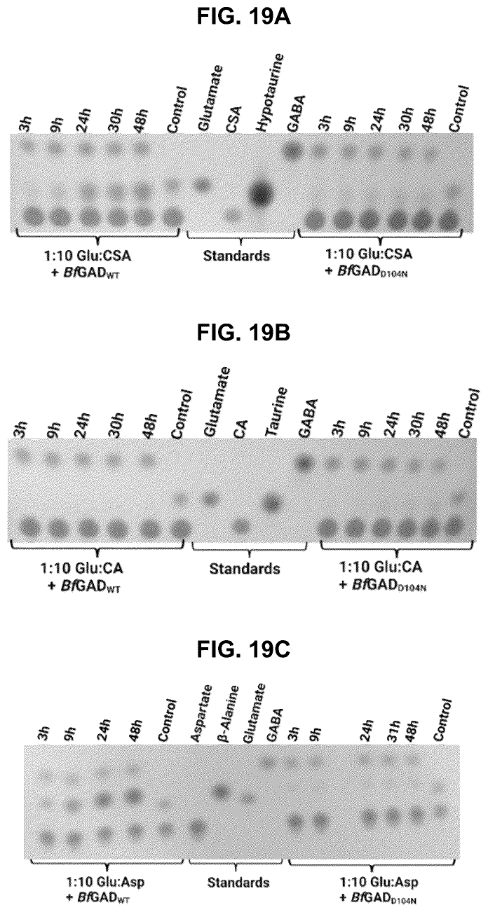

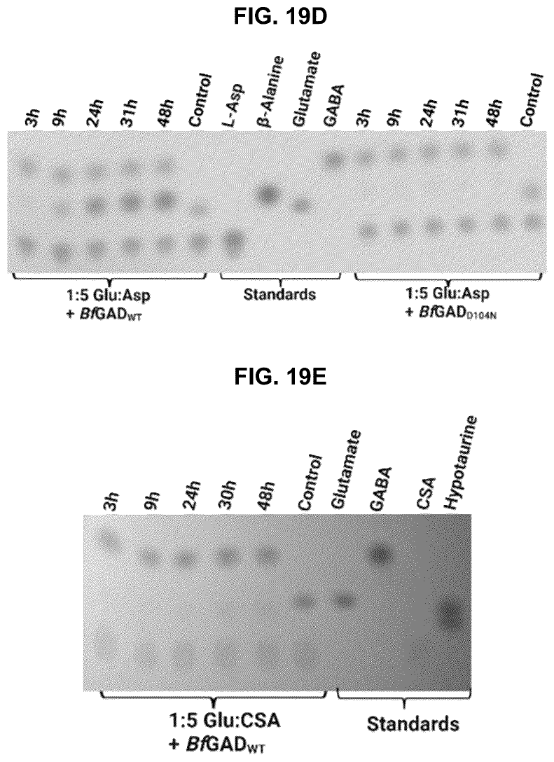

In a complex gut environment, the organism will encounter multiple substrates simultaneously. To investigate how BfGAD_{WT} functions in such a complex environment, competition assays were conducted between various substrates. To this end, detection of the presence of decarboxylated products in mixtures containing the native substrate L-glutamate was attempted with a specific alternate substrate. The data with BfGAD_{WT} indicated that when the concentration ratios of the native substrate to the alternative substrate were 1:1 and 1:2.5, GABA was predominantly seen as the primary decarboxylated product (data not shown). However, when the concentration ratios were changed to 1:5 (L-Glu with L-CSA/L-Asp) and 1:10 (L-Glu with L-CSA/L-CA/L-Asp), a gradual increase over time in the formation of the alternative decarboxylated products-hypotaurine, taurine, and β-alanine was observed (FIG. 5-6). For the BfGAD_{WT} catalyzed decarboxylation reactions of L-CSA and L-Asp, the resulting products hypotaurine and β-alanine were observed when the alternate/these substrates were in 5-fold excess of/to the native substrate L-glutamate. However, the accumulation of products hypotaurine and β-alanine becomes significant only after 9 hours (for β-alanine)—24 hours (for hypotaurine) (FIG. 19D-E). In contrast, when L-CSA and L-Asp are used in 10-fold excess of L-glutamate, the resulting products, hypotaurine and β-alanine, started accumulating significantly much earlier around 3 hours (FIG. 5, FIG. 6B). The accumulation of taurine was not observed even when 10-fold excess of L-CA was mixed with L-glutamate in the BfGAD_{WT} catalyzed reaction (FIG. 6A). However, as mentioned above, CO₂ was detected in the same time frame (48 hours) in the reaction catalyzed by BfGAD_{WT} when L-CA was provided as the sole substrate (FIG. 16A). The inability to detect taurine might have been due to the low concentrations produced under the competitive environment of the mixed substrate pool. Unlike BfGAD_{WT}, BfGAD_{D104N} was able to decarboxylate and accumulate taurine to a small extent when L-CA was mixed with L-glutamate at a 10-fold excess concentration, especially between 24-48 hours (FIG. 6A).

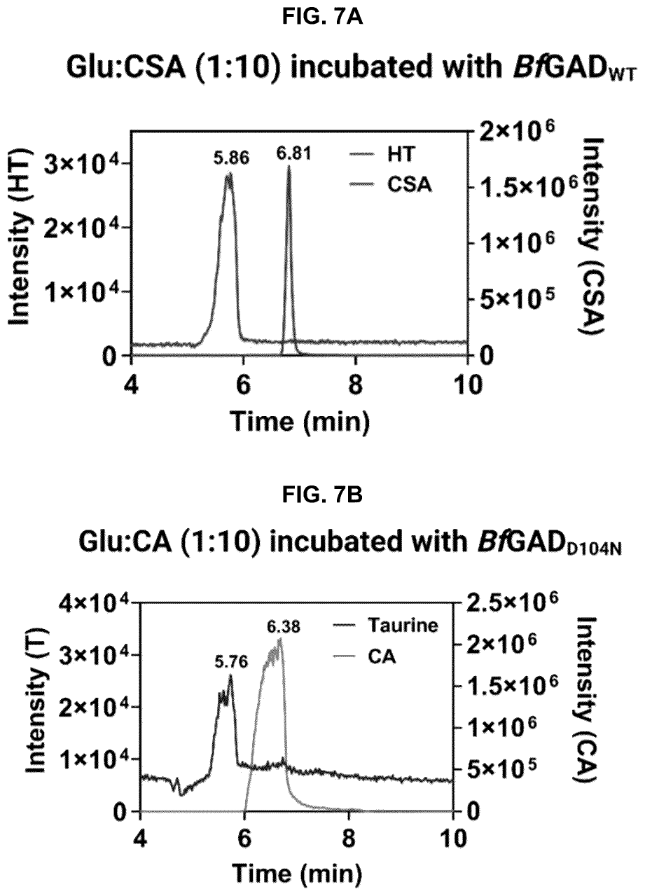

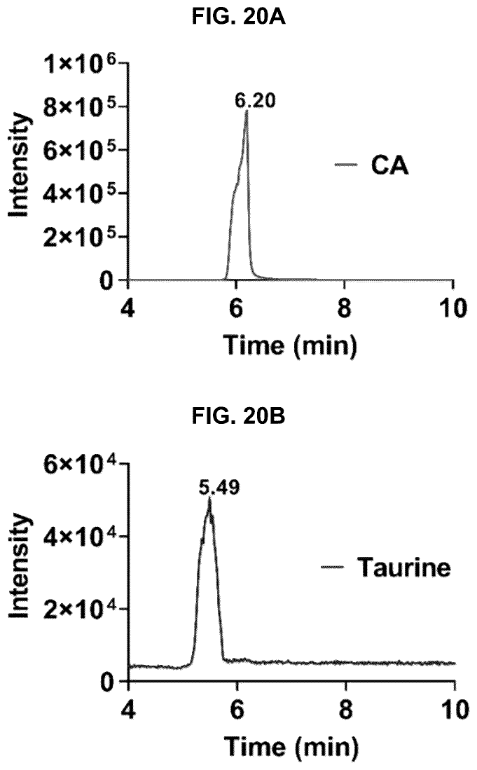

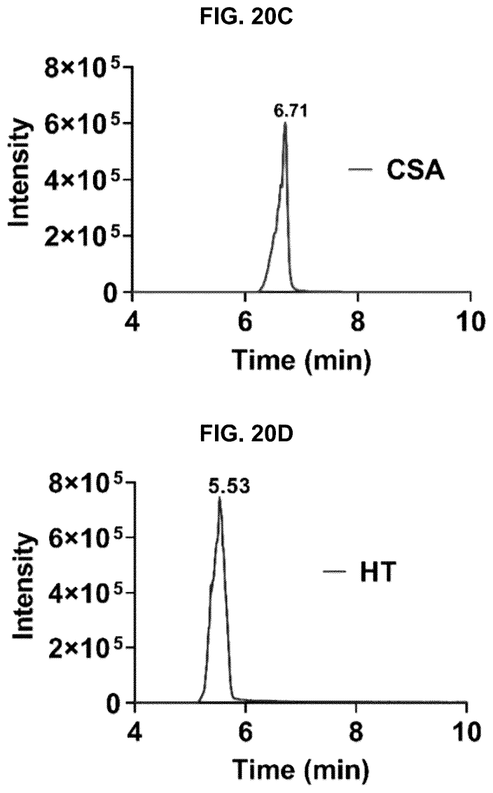

The decarboxylated products hypotaurine and taurine were analyzed using LC-ESI-MS/MS in addition to TLC to confirm their presence and mass in mixed substrate assays where L-glutamate to L-CSA or L-CA ratios were 1:10. The qualitative analysis of the amino acids was conducted using MRM mode by running mixed standards (FIG. 20). FIG. 7A depicts the presence of the product hypotaurine and the leftover/remaining L-CSA substrate in the mixed substrate reactions with a 1:10 ratio of L-glutamate:L-CSA catalyzed by BfGAD_{WT}. FIG. 7B depicts the presence of taurine and leftover/remaining L-CA in the mixed substrate reactions with a 1:10 ratio of L-glutamate:L-CA catalyzed by BfGAD_{D104N}. The native substrate and product, L-glutamate and GABA, were not analyzed in these samples using LC-MS/MS because the presence of these molecules was verified/confirmed by both TLC and GABase assay prior to LC-MS/MS analysis.

Prior research has implicated gut microbial contributions to the production of γ-aminobutyric acid (GABA), specifically from Bacteroides genus. GABA, a neurotransmitter, is modulated in many neurodegenerative diseases, including Alzheimer's and dementia, where lower GABA levels have been consistently reported. Microbes from the genus Bacteroides are known to be fluctuated in individuals afflicted by Alzheimer's and dementia. Due to the prevalent nature of Bacteroides in the gut and the modulation of Bacteroides during Alzheimer's, the mechanism by which GABA is produced in these organisms was investigated. For this reason, an annotated glutamate decarboxylase from B. fragilis (BfGAD) was selected as a candidate enzyme representing annotated GADs in all Bacteroides.

Most of the studied microbial GADs are oligomeric which include dimeric, tetrameric or hexameric states. It is known that the functional GAD unit is a dimer as residues from both monomers make an intact active site and play an important role during the catalysis. Therefore, an oligomeric composition that is not an even number would be unusual in this context. The gel filtration chromatography data from pH 4.7 buffer gave a molecular weight of the protein that is higher than a dimer but lower than a tetramer. It was hypothesized that the protein likely exists in an equilibrium between these two states, with a tendency towards a tetrameric state. However, at pH 7.2, there might be a shift in this equilibrium favoring a more dimeric state. This shift is reflected in the observed molecular weight, which is closer to that of a dimer than a tetramer at pH 7.2. This shift in oligomeric states has been observed previously in E. coli GAD that undergoes significant changes in oligomeric forms upon shift in the pH. Thus, purified BfGAD_{WT} behaves similarly in the oligomeric composition to other characterized prokaryotic GADs.

However, the kinetic parameters of BfGAD_{WT} vary from previously investigated prokaryotic GADs. It was found that BfGAD_{WT} is an allosteric enzyme, displaying a positive cooperativity with the substrate L-glutamate. This happens when binding of substrate on one site or monomer of the protein facilitates or promotes the binding of additional substrate to the other sites or monomers of the protein. Instead of a hyperbolic dependence, a sigmoidal curve is observed in the plot of initial velocity versus substrate concentration. This phenomenon has not been seen with any other prokaryotic GADs. The physiological consequences of this cooperativity in BfGAD need to be explored further. Cooperative binding and adaptability in the allosteric enzymes help amplify the enzyme's response to changes in substrate concentration, making it more sensitive to the physiological conditions.

Although the kinetic parameters are different for the BfGAD_{WT}, there are prominent structural similarities among BfGAD_{WT} and other studied microbial GADs. AlphaFold model of BfGAD predicts active site architecture to be very similar to other prokaryotic GADs. Particularly, catalytic residues in the active site are positioned similar to those in EcGAD and BfGAD, suggesting that corresponding residues in BfGAD might maintain similar interactions with ligands. While the hydrogen bond interactions of the residues with the substrate analog glutarate are predicted to be within the expected limits in the superimposed structures of EcGAD and BfGAD_{WT}, there are differences in the hydrogen bond lengths when amino acid substitutions are made in the active site. The changes in these H-bonding interactions could potentially be involved in some capacity in accommodating or excluding alternative substrates in the active site of the engineered BfGADs to evolve new activity or specificity.

In contrast to GADs from Bacteroides sp., microbial GADs from various other genera have been widely characterized. Most of these studies show that many annotated microbial glutamate decarboxylases prefer L-glutamate as a substrate. However, substrate promiscuity has been seen in GADs from different domains of life. For example, E. coli GAD was able to convert a phosphinic analog of glutamate to a phosphinic analog of GABA. In addition, human and other mammalian GADs are known to catalyze decarboxylation of substrates other than L-glutamate. Specifically, sulfinic acid and sulfonic acid derivatives of alanine (known as cysteine sulfinic acid and cysteic acid respectively) are known alternative substrates for mammalian GADs.

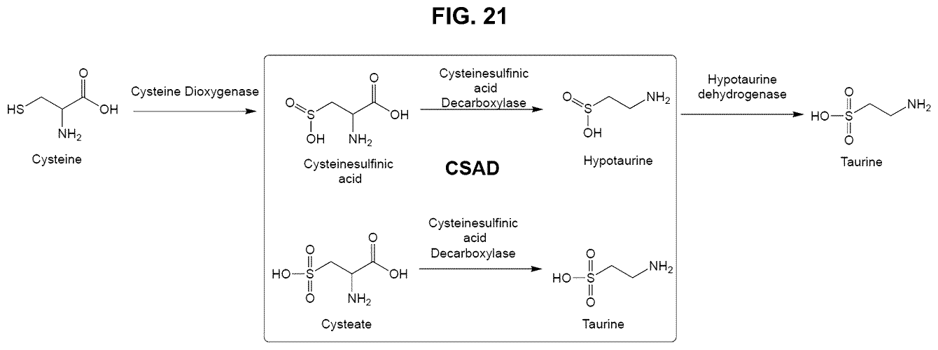

Decarboxylation of these molecules by mammalian GADs generate hypotaruine and taurine, from the respective sulfinic or sulfonic acids. Additionally, mammals have a couple of other de novo pathways for the production of taurine and hypotaurine. The primary pathway involves the enzyme cysteine sulfinic acid decarboxylase (CSAD) that generates taurine and its intermediate hypotaurine by decarboxylating either L-cysteic acid (CA) or L-cysteine sulfinic acid (CSA). In addition to hCSAD, mammalian GADL1 (glutamic acid decarboxylase like 1) enzyme that has very high sequence similarity with hCSAD, is capable of producing taurine. Taurine, a sulfur containing non-proteinogenic amino acid whose production is controlled by the enzyme cysteine sulfinic acid decarboxylase (CSAD). However, except for a few marine microbes the major de novo pathway for taurine formation with the help of CSAD has not been seen in other prokaryotes. Interestingly, in these microbes CSAD genes are present in the operon containing cysteine dioxygenase (CDO) enzyme that catalyzes the conversion of L-cysteine to L-cysteine sulfinic acid (L-CSA) which then can be converted to hypotaurine and taurine (FIG. 21). CDOs are not present in the members of the human gut microbiome possibly due to the hypoxic and anaerobic conditions of the gut. Despite an extensive bioinformatics search, no gut microbial genes were found to be annotated as CSAD (cysteine sulfinic acid decarboxylase). Due to the lack of the annotated CSAD enzymes responsible for the de novo taurine biosynthesis, the role of BfGAD in the formation of taurine and its derivatives in addition to GABA was investigated.

Apart from taurine, mammalian GADL1 is also able to generate β-alanine from L-aspartate. β-alanine is a precursor for the dipeptide beta-alanyl-L-histidine in humans, commonly known as carnosine. Carnosine is found in muscles and brain tissues at high concentrations. In microbes, β-alanine is a precursor for coenzyme A (CoA) biosynthesis, which is an important molecule in various metabolic pathways. Both carnosine and β-alanine show protective effects in individuals with cognitive deficits and Alzheimer's. Moreover, higher serum concentrations of β-alanine prevent dementia. Considering the structural similarities between L-glutamate and L-aspartate and the ability of some GADs to use L-aspartate as a substrate, the possible role of BfGAD in the production of β-alanine via the decarboxylation of L-aspartate was examined. The neuromodulatory molecules, GABA, taurine, and β-alanine generated by glutamate decarboxylases or enzymes similar to GADs, have the ability to reverse cognitive deficits in neurodegenerative disorders like dementia and Alzheimer's. This prompted the need to understand the modulation of these molecules by the members of the human gut microbiome.

These results show that the BfGAD_{WT} is promiscuous and is able to decarboxylate four additional substrates structurally similar to L-glutamate to produce hypotaurine, taurine, homotaurine, and β-alanine. There are distinct enzymes in microbes such as aspartate1-decarboxylase (A1DC) and aspartate 4-decarboxylase (A4DC) that catalyze decarboxylation of L-aspartate to produce β-alanine and L-alanine respectively. While the genome of B. fragilis has genes annotated for both these enzymes, it is interesting to observe that the glutamate decarboxylase from B. fragilis still shows decarboxylation activity towards L-aspartate to produce β-alanine. Additionally, prokaryotic A1DC and eukaryotic A1DC show evolutionary divergence where the former uses pyruvoyl cofactor whereas the latter uses PLP cofactor. Thus, BfGAD_{WT} and variants harbor an activity that is mostly seen in eukaryotic organisms.

To understand the factors that drive substrate specificity in BfGAD_{WT} and to evolve enzymes that potentially have switched substrate preferences or specificity, two active site residues were chosen that are known to make interactions with bound ligands. Headspace GC was used for the detection of evolved CO₂ which is the common decarboxylation product for all enzyme catalyzed reactions (WT and engineered enzymes). Absolute quantification of CO₂ (g) was challenging due to its high solubility at acidic pH where the enzymatic reactions were conducted. In these conditions, the CO₂ produced through the reaction might still be in a soluble form as a dissolved CO₂. So, headspace GC was utilized as a primary tool to identify alternate substrates by allowing detection of CO₂ peak that served as a positive indication of enzymatic decarboxylation of the substrate molecules. From the identification provided by the GC experiments, subsequent analysis was carried out with TLC to detect corresponding decarboxylated products from the multiple substrates. The headspace GC data reinforce the TLC data in most cases. Both engineered enzymes BfGAD_{D104N} and BfGAD_{F81W} retain preference for L-glutamate as a substrate. However, their preferences for alternate substrates are different than the BfGAD_{WT}. Additionally, significant spectral perturbations are observed with engineered GADs indicating that the local environment of the cofactor binding site is changed due to those amino acid substitutions. With this, it was shown that there may be a de novo pathway in gut microbes to make taurine and BfGAD_{WT} is able to produce multiple neuromodulatory molecules.

### Example 2

Generation of a Bacteroides fragilis Strain with the Deletion of the Gene for Glutamate Decarboxylase (B. fragilis ΔGAD) and Testing

The bacterial strains and plasmids used are provided in Table 4.

TABLE 4
Bacteria, Plasmids, and Growth Conditions for GAD Deletion Mutant
Production

| Strain | Description | Growth Conditions |
|---|---|---|
| B. fragilis NCTC 9343 | Type strain | BHIS, 37° C., anaerobic |
| pLGB13 | Suicide vector for allelic replacement in | LB, 37° C., 100 μg/mL |
| | Bacteroides, erythromycin selection and | Ampicillin, aerobic |
| | aTC-inducible ss-Bfe1 counterselection | |
| S17-λpir | E. coli competent cells | LB, 37° C., aerobic |

BHIS - brain heart infusion media, supplemented with Hemin, Vitamin K1, and sodium polyanetholesulfonate (SPS)
LB - Luria-Bertani medium

### Protocol for Mutant Generation

To generate mutant (B. fragilis ΔGAD), plasmid pLGB13 was digested using EcoRV and Pstl. A 2.5 kb fragment upstream and downstream of GAD was amplified with overhangs of the digested suicide vector pLGB13. The upstream and downstream regions of the gene were ligated into the digested pLGB13 plasmid using Hi Fi DNA assembly. The plasmid was transformed into S17-Apir competent cells and the colonies were screened for clones. The presence of the plasmid and gene were confirmed by restriction digestion analysis and sequencing.

Liquid overnight cultures of donor strains (E. coli S17-Apir carrying the plasmid of interest) were grown aerobically, and the recipient strain (B. fragilis) was grown anaerobically until E. coli reached an OD600 of 0.3, and B. fragilis reached an OD600 of 0.04. A 100 mL sample of B. fragilis (OD600=0.04) and 10 mL sample of E. coli (OD600=0.3) were separately centrifuged at 9000×g for 10 min. The pellets were resuspended and washed twice in 1×PBS and finally resuspended in 1×PBS. A 0.5 mL aliquot of each resuspended pellet was pipetted directly on to the center of a Brain-Heart Infusion media (BHI) plate without spreading or streaking. The plates were incubated aerobically at 37° C. overnight (agar facing up).

After overnight incubation, the bacterial patch was scraped with an inoculation loop and streaked on BHI plate containing gentamicin (200 μg/mL, to kill the E. coli) and erythromycin (25 μg/mL, to select for the cointegrate or transconjugant strains) and incubated anaerobically at 37° C. for up to 2-3 days.

Colonies were picked after 2-3 days and streaked on fresh plates for isolation. PCR analysis for the first recombinants was performed using Phusion DNA polymerase with the ampicillin primers (900 bp). After confirming first recombinants, a single colony was grown in 10 mL of BHI, overnight, 37° C., anaerobically. The overnight culture was diluted from 10⁻¹ to 10⁻⁸ and plated on BHI plates containing 100 ng/ml aTC. These plates were incubated at 37° C., anaerobically for 48 hours. The observed colonies were a combination of WT and mutants. A sample of colonies was picked, and PCR amplification was performed to confirm the mutants.

BHI cultures were started from single colonies of B. fragilis WT and ΔGAD. After 48 hours, subcultures of 10 mL for each were started. After 2 days, the cells were centrifuged at 4000×g for 30 min. The supernatant was discarded, and the pellets were washed twice with 1×M9 media. After that, cell pellets were resuspended in the minimal media cultures with and without L-glutamate to observe the production of GABA. After 48 hours, the supernatants from the cultures were run on a TLC (thin-layer chromatography) plate to observe if GABA was produced and, if so, exported outside the cell.

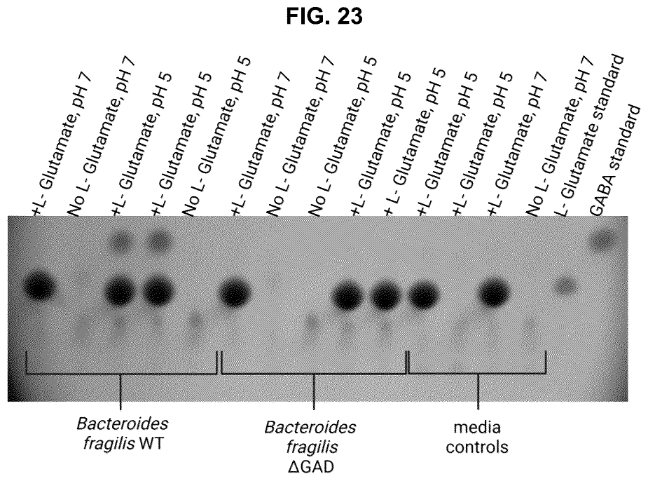

FIG. 23 shows that GABA exported from the cells was observed only with B. fragilis WT, not in the B. fragilis ΔGAD mutant, thus confirming the deletion of the GAD gene responsible to produce GABA in Bacteroides fragilis.

What is claimed:

1. An isolated polypeptide comprising a Bacteroides fragilis glutamate decarboxylase (BfGAD) mutant having one or more amino acid substitutions introduced into a wild-type BfGAD polypeptide sequence (SEQ ID NO: 2), where the BfGAD mutant provides an improvement in decarboxylase activity as compared to the wild-type BfGAD polypeptide sequence (SEQ ID NO: 2).

2. The isolated polypeptide of claim 1, wherein the BfGAD mutant comprises one or more of the following substitutions: D104N or F81W relative to the wild-type BfGAD polypeptide sequence (SEQ ID NO: 2).

3. The isolated polypeptide of claim 1, wherein the BfGAD mutant comprises an amino acid sequence having at least 95% identity to SEQ ID NO: 4 or SEQ ID NO: 6.

4. The isolated polypeptide of claim 1, wherein the BfGAD mutant comprises the amino acid sequence of SEQ ID NO: 4 or SEQ ID NO: 6.

5. An isolated nucleotide encoding a Bacteroides fragilis glutamate decarboxylase (BfGAD) mutant having one or more amino acid substitutions introduced into a wild-type BfGAD polypeptide sequence (SEQ ID NO: 2).

6. The isolated nucleotide of claim 5, wherein the BfGAD mutant comprises one or more of the following substitutions: D104N or F81W relative to the wild-type BfGAD polypeptide sequence (SEQ ID NO: 2).

7. The isolated nucleotide of claim 5, wherein the isolated nucleotide comprises a polynucleotide sequence having at least 95% identity to SEQ ID NO: 3 or SEQ ID NO: 5.

8. The isolated nucleotide of claim 5, wherein the isolated nucleotide comprises the polynucleotide sequence of SEQ ID NO: 3 or SEQ ID NO: 5.

9. An isolated nucleotide vector comprising the isolated nucleotide of claim 5.

10. A cell comprising the isolated nucleotide vector of claim 9.

11. A method for synthesizing neuromodulatory molecules, the method comprising:
contacting a Bacteroides fragilis glutamate decarboxylase (BfGAD) mutant with a substrate, and
obtaining one or more neuromodulatory molecules.

12. The method of claim 11, wherein the BfGAD mutant comprises one or more of the following substitutions: D104N or F81W relative to a wild-type BfGAD polypeptide sequence (SEQ ID NO: 2).

13. The method of claim 11, wherein the BfGAD mutant comprises an amino acid sequence having at least 95% identity to SEQ ID NO: 4 or SEQ ID NO: 6.

14. The method of claim 11, wherein the BfGAD mutant comprises the amino acid sequence of SEQ ID NO: 4 or SEQ ID NO: 6.

15. The method of claim 11, wherein the neuromodulatory molecules comprise γ-aminobutyric acid (GABA), hypotaurine, taurine, homotaurine, or β-alanine.

16. The method of claim 11, wherein the substrate comprises L-glutamate, L-cysteine sulfinate, L-cysteate, L-homocysteate, or L-aspartate.

17. A neuromodulatory molecule comprising γ-aminobutyric acid (GABA), hypotaurine, taurine, homotaurine, or β-alanine synthesized by the method of claim 11.

18. A method for creating a Bacteroides fragilis strain with a deletion of the gene encoding glutamate decarboxylase (B. fragilis ΔGAD), the method comprising:
(a) transforming a suicide vector encoding B. fragilis glutamate decarboxylase into competent E. coli cells and screening for transformants;
(b) independently growing liquid cultures of transformed E. coli aerobically and growing liquid cultures of B. fragilis anaerobically;
(c) independently isolating cells from the liquid cultures of the transformed E. coli and B. fragilis and resuspending the cells in a volume of liquid;
(d) plating aliquots of each of the resuspended transformed E. coli and B. fragilis cells on a culture media plate and incubating aerobically at 37° C. overnight;
(e) isolating B. fragilis cells from the culture media plate and streaking the cells on a second culture media plate comprising gentamicin and erythromycin and incubating the second culture media plate anaerobically at 37° C. for 2-3 days;
(f) selecting individual B. fragilis colonies and analyzing the colonies for a deletion of the gene encoding glutamate decarboxylase; and
(g) identifying a B. fragilis cell having a deletion of the gene encoding glutamate decarboxylase.
